(19) United States
(12) Patent Application Publication
(10) Pub. No.: US 2023/0279465 A1
(43) Pub. Date: Sep. 7, 2023

(54) METHODS OF ANCHORING FRAGMENTED NUCLEIC ACID TARGETS IN A POLYMER MATRIX FOR IMAGING

(71) Applicant: Vizgen, Inc., Cambridge, MA (US)

(72) Inventors: Jiang He, Cambridge, MA (US); Lizhi He, Cambridge, MA (US); Arpan Ghosh, Cambridge, MA (US)

(21) Appl. No.: 18/111,070

(22) Filed: Feb. 17, 2023

Related U.S. Application Data
(60) Provisional application No. 63/311,319, filed on Feb. 17, 2022.
(60) Provisional application No. 63/424,891, filed on Nov. 12, 2022.

(51) Int. Cl. C12Q 1/6806; C12Q 1/6876; C12Q 1/6841

(52) U.S. Cl. CPC C12Q 1/6806; C12Q 1/6876; C12Q 1/6841

(57) ABSTRACT

The present disclosure is generally directed to methods for anchoring nucleic acid in a matrix and subsequently imaging nucleic acid targets (e.g. RNA transcript molecules) within tissue samples, for example, formalin-fixed paraffin-embedded (FFPE) tissue sections wherein the nucleic acid may be fragmented. For example, a method of anchoring target nucleic acid within a matrix and clearing non-target cellular components is provided herein, and the method includes contacting a tissue sample with at least two anchoring agents, wherein the first anchoring agent forms a covalent bond with the target nucleic acid and the second anchoring agent contains an oligonucleotide that hybridizes with the target nucleic acid; embedding the sample in a polymer matrix wherein the first and second anchoring agents each form a covalent bond with the polymer matrix; and, clearing the non-target cellular components from the polymer matrix wherein the target nucleic acid remains anchored in the polymer matrix to form a matrix anchored target nucleic acid sample. Additional steps include contacting the anchored target nucleic acid sample with one or more primary oligonucleotide probes that hybridize to the target nucleic acids and a plurality of secondary nucleic acid probes containing a fluorescent label and a recognition sequence that hybridizes to a sequence of the primary nucleic acid probe and imaging the target nucleic acids.

### RELATED APPLICATIONS

This application claims priority to provisional application Nos. U.S. Ser. No. 63/311,319 filed 17 Feb. 2022; and, U.S. Ser. No. 63/424,891 filed 12 Nov. 2022, each of which are hereby incorporated into this application in their entirety.

### FIELD OF THE DISCLOSURE

This application relates generally to the field of in situ imaging, and in particular, relates to methods of determining nucleic acid targets within tissue samples.

### BACKGROUND OF THE DISCLOSURE

To accurately profile the gene expression in tissue samples in situ, a spatial transcriptomics technique with high detection efficiency and single molecule resolution is required. In situ single cell transcriptomic imaging technology, such as Multiplexed Error-Robust Fluorescence In Situ Hybridization (MERFISH), enables the direct profiling of the spatial organization of intact tissue with subcellular resolution.

However, background—from factors such as cellular autofluorescence—often make in in situ imaging applications in tissue samples more challenging. Without wishing to be bound by any theory, it is believed that certain components such as proteins and lipids, unbound or irrelevant nucleic acids, fluorescent components (bleached or unbleached), or the like may create problems in imaging or analysis, e.g., due to autofluorescence, components that quench fluorescent molecules, off-target binding, or other phenomena. For example, it is believed that nucleic acid probes may not bind to a proper target within a sample, and instead may bind “off-target” to other cellular components, including but not limited to proteins, lipids, RNA, DNA, etc. Similarly, probes targeting one DNA or RNA molecule may bind “off-target” to the wrong DNA or RNA molecule. These interactions could be driven, for example, by imperfect base pairing, charge-charge interactions, or other molecular interactions. Accordingly, a polymer matrix or gel may be applied to a sample to immobilize desired nucleic acid molecules (or other desired targets), while the components (“non-target cellular components”) to which nucleic acid probes bind off-target can be removed or degraded from the sample. This may reduce the amount of probes that bind off-target, facilitating imaging or other analysis of the sample. Other components, such as proteins and lipids, may be removed or degraded from the sample. This may reduce the amount of background, facilitating imaging or other analysis of the sample. The methodologies developed for removing cellular components from a gel embedded tissue section work sufficiently well with fresh and fixed frozen samples wherein anchoring oligonucleotides and target probes are added to a sample, then embedded in a gel and the cellular components cleared or removed followed by imaging (“Protocol B”). See FIG. 1. That method as provided herein, when used with formalin fixed paraffin embedded (“FFPE”) tissue sections is insufficient for immobilizing the target nucleic acid and for hybridization of a target probe to nucleic acid for imaging.

For example, the methods for preparation of samples for MERFISH imaging are disclosed in US Patent Publ. 2019/0264270, entitled “Matrix Imprinting and Clearing” (the contents of which are incorporated herein by reference). However, MERFISH imaging has been previously demonstrated in fresh and fixed frozen tissue blocks, and not previously in formalin-fixed paraffin-embedded (FFPE) tissue sections. Indeed, while FFPE tissue sections are the most widely used clinical sample types in histology and molecular diagnosis, FFPE samples are known as often not compatible with in situ single-cell transcriptomic analysis due to RNA degradation and protein crosslinking. Therefore, there is a need for methods and/or reagents that allow the in situ single-cell transcriptomic analysis from FFPE tissue samples.

Accordingly, the present disclosure provides improved methods of imaging nucleic acid targets including preparation of tissue samples that allows in situ single-cell transcriptomic imaging (e.g., MERFISH) from FFPE tissue section.

### SUMMARY OF THE DISCLOSURE

Described herein are methods and reagents thereof for in situ single-cell transcriptomic analysis from FFPE tissue sections, or other tissue samples wherein nucleic acid fragmentation is suspected. In embodiments provided herein is a method for anchoring and imaging target nucleic acid molecules (e.g., mRNA transcripts) from a FFPE tissue sample comprising: a) contacting the tissue sample with at least two anchoring agents, wherein the first anchoring agent forms a covalent bond with a target nucleic acid and the second anchoring agent comprises an oligonucleotide that hybridizes with the target nucleic acid; b) embedding the sample in a polymer matrix wherein the first and second anchoring agents each form a covalent bond with the polymer gel; c) clearing the non-target cellular components from the polymer matrix wherein the target nucleic acid remains anchored in the polymer matrix to form a matrix anchored target nucleic acid sample. In certain embodiments, the methods further comprise an imaging step d) contacting the anchored target nucleic acid sample with one or more primary oligonucleotide probes that hybridize to the target nucleic acids and a plurality of secondary nucleic acid probes comprising a fluorescent label and a recognition sequence that hybridizes to a sequence of the primary nucleic acid probe and imaging the target nucleic acids.

### BRIEF DESCRIPTION OF THE DRAWINGS

FIG. 1 shows the comparison of Protocol A (methods of this disclosure) and Protocol B (comparative protocol). See Example 1.

FIGS. 2A-2C show MERFISH imaging with 128-plex gene panel in FFPE mouse small intestine tissue sections according to Example 1 (Protocol A). FIG. 2A shows spatial distribution of select genes across the tissue; FIG. 2B shows tissue morphology visualized by select transcripts (Left) and distribution of all transcripts in zoomed in region; FIG. 2C shows correlation of MERFISH counts with bulk RNA sequencing FPKM (fragments per kilobase million) data, indicating the measurement is quantitative and highly accurate. Scale bar: 1 mm. See Example 2.

FIGS. 3A-3C show MERFISH imaging with 244-plex gene panel in FFPE human colon cancer tissue sections according to Example 1 (Protocol A). FIG. 3A) Spatial distribution of select genes across the tissue; FIG. 3B) Tissue morphology visualized by select transcripts (Left) and distribution of all transcripts in zoomed in region; FIG. 3C) Correlation of MERFISH counts with bulk RNA sequencing FPKM data. Scale bar: 1 mm. See Example 2

FIGS. 4A-4C show MERFISH imaging with 483-plex gene panel in FFPE mouse brain tissue sections according to Example 1 (Protocol A). FIG. 4A) Spatial distribution of select genes across the tissue; FIG. 4B) Tissue morphology visualized by select transcripts (Left) and distribution of all transcripts in zoomed in region; FIG. 4C) Correlation of MERFISH counts with bulk RNA sequencing data. Scale bar: 1 mm. See Example 2

FIG. 5 shows comparison of the sample preparation according to the protocol of Example 1 (“Protocol A”) to a comparative protocol (“Protocol B”). For Protocol B, the MERFISH probes (e.g., primary oligonucleotide probe) and anchor probs are added prior to tissue clearing step. FFPE mouse small intestine samples were processed with Protocol A or Protocol B, fresh frozen mouse small intestine samples were processed with Protocol B. Average counts of transcripts per field of view (FOV) for both conditions are shown. N-3. See Example 3 and FIG. 1.

FIGS. 6A-6C shows MERFISH imaging in 15 different archival human FFPE tissue section samples with 244-plex gene panel. For each dataset, 1000-2000 fields of views were captured, generating 10s-100s million counts per tissue slice. FIG. 6A) Average MERFISH counts per field of view with an area size of 200×200 μm, indicating the workflow works robustly across a wide range of human normal and cancer FFPE tissue section samples. FIG. 6B) MERFISH data quality is correlated with sample's RNA quality, as indicated by DV200 value above 40%. DV200 is the percent of RNA fragments >200 nucleotides in samples. FIG. 6C) Correlation of MERFISH counts with bulk RNA sequencing FPKM data across different tissues. See Example 4.

FIG. 7 shows comparison of the samples prepared according to the protocol of Example 1 (“Protocol A”) to a comparative method (“Protocol B”) across different sample types showing an average counts per field of view with an area size of 200×200 μm across different tissue types and a correlation of MERFISH data, with correlation co-efficient included for each tissue type. See Example 5.

FIG. 8 Shows protocol A outperforms protocol B in a variety of fresh frozen (FF) human samples, while maintaining the accuracy of measurement. FIG. 8A) Fresh frozen human lymph node, lung, colon and kidney samples were processed with Protocol A (anchor target nucleic first and hybridize after polymer matrix embedding and clearing of non-target cellular components) or Protocol B with a panel of 244 genes. B, and all samples were then imaged on MERSCOPE. Average counts of transcripts per field of view (FOV) for both conditions are shown.

FIG. 9 Shows protocol A outperforms protocol B in a variety of fresh frozen (FF) human samples, while maintaining the accuracy of measurement, wherein the imaged transcripts per gene with Protocol A were correlated with Protocol B in human lung and kidney samples. The correlation coefficients are 0.99 and 0.98 respectively, indicating that Protocol A was able to recapitulate the expression level measured by Protocol B and overall there was substantially more counts with Protocol A.

FIG. 10A-C show that the FFPE workflow is highly sensitive, accurate and reproducible. FIG. 10A shows the correlation of MERSCOPE data between two human ovarian cancer slices from the same patient. Correlation coefficient is 0.99, indicating the measurement is highly reproducible. In FIG. 10B, human ovarian cancer sample 1 was analyzed by MERSCOPE using a 500 gene panel, and adjacent slices were analyzed by bulk RNA sequencing. Correlation analysis between MERFISH counts and FPKM values from bulk RNA sequencing is shown. The correlation coefficient is 0.82, indicating the measurement is highly accurate. FIG. 10C presents a correlation analysis between MERSCOPE data and bulk RNA sequence was performed across 14 cancer samples, and correlation co-efficient show high accuracy across multiple cancer types and replicates. See Example 8.

FIGS. 11A-F show that FFPE cell segmentation workflow enables true atlasing in dense tissue. In FIG. 11A, FFPE human liver cancer was immunostained with a cell boundary staining kit and DAPI for nucleus staining. In FIG. 11B, deep learning-based cell segmentation algorithm was used to segment cells. The polygon masks for each identified cell are shown. FIG. 11C shows UMAP visualization of 17 different cell types identified in human liver cancer generate from MERFISH transcript data. FIG. 11D shows the spatial distribution of identified cell types across the tissue in boxed region from FIG. 11B. FIG. 11E shows spatial distribution of fibroblasts in boxed region from FIG. 11B. Fibroblast marker gene COL1A1 (shown in yellow). FIG. 11F shows the partial distribution of endothelial cells in boxed region from B. Endothelial marker gene PECAM1 shown in green. See Example 8.

FIG. 12 shows the spatial distribution of identified cell types across different FFPE tumor samples. Different cancer samples, including breast cancer, colon cancer, melanoma, lung cancer, liver cancer, ovarian cancer, prostate cancer and uterine cancer, were analyzed by MERSCOPE using a 500 gene panel, together with cell boundary staining kit to label the cell boundary. Cells are segmented and subjected for single cell analysis. Identified cells in each sample are colored to show the spatial distribution of different cells across the sample. Scale bar: 1 mm. See Example 8.

FIG. 13 shows that the FFPE protocol can be used to show the spatial distribution of the expression of select genes (ACTA2, CD3D, LGR5, MK167, and PECAM1) in human breast cancer. FIG. 13A shows the spatial distribution of select genes including ACTA2 (green), CD3D (red), LGR5 (light green), MKI67 (magenta) and PECAM1 (blue) from 500 genes analyzed across the tissue. Scale bar: 1 mm. FIG. 13B provides a zoomed-in view of the boxed region in FIG. 13A. Scale bar: 1 mm. FIG. 13C shows a zoom-in view of the boxed region in FIG. 13B, with cell boundary polygon masks shown in grey. Scale bar: 250 μm. See Example 8

FIG. 14A-E show that the FFPE protocol can be used for cell type identification and mapping in human breast cancer. FIG. 14A provides UMAP visualization of different cell types identified in human breast cancer generated from MERFISH transcript data. FIG. 14B shows the spatial distribution of 14 identified cell types across the tissue. FIG. 14C shows the spatial distribution of identified cell types in boxed region in FIG. 14B. FIG. 14D shows the spatial distribution of two types of fibroblasts (fibroblast 1 in green and fibroblast 2 in red) in boxed region in FIG. 14C. Both types of fibroblasts express COL1A1 gene, while fibroblast 2 expresses proliferation marker MKI67. FIG. 14E provides a dot plot showing the marker genes for each cell type. See Example 8

FIG. 15 shows that the FFPE protocol can be used to characterize immune cell types in the tumor microenvironment. FIG. 15A shows the T/NK cell cluster from a breast cancer sample was selected for sub-clustering analysis. UMAP visualization of sub-clustering analysis showing 7 different immune cell subtypes within human breast cancer. FIG. 15B provides a dot plot showing the marker genes for each immune cell type, including Myeloid cells, CD4+ T cells, CD8+ T cells, CD4+ regulatory T cells (Tregs), and NK lineage cells. FIG. 15C provides the spatial distribution of Tregs. FIG. 15D provides the spatial distribution of CD4+ T cells. FIG. 15E provides the spatial distribution of select genes within a magnified region in human breast cancer, with CD4, CD8A, FOXP3, NCR1 and CTLA4 shown. Note that FOXP3 positive Tregs expresses T cell exhaustion marker. See Example 8

### DETAILED DESCRIPTION OF THE DISCLOSURE

The present disclosure generally relates to preparation of tissue samples for in situ imaging when fragmentation of the nucleic acid, especially mRNA, is suspected. In embodiments the sample is a FFPE tissue sample. The present disclosure also provides preparation of tissue samples that allows in situ single-cell transcriptomic imaging (e.g., MERFISH, smFISH) to detect nucleic acid targets in the samples. The methods of the present disclosure can be used for the preparation of gene expression profiles of tissue samples. Other aspects are generally directed to systems or kits involving such methods or the like.

In certain embodiments, provided herein, are methods, and compositions thereof, for FFPE sample preparation to be used for transcriptome analysis. In embodiments, transcriptome analysis includes single molecular (sm)FISH, barcoding (also referred to herein as “codewords”) methods to quantify transcripts transcriptome wide, combinatorial barcoding methods for quantitative and spatial transcriptome analysis (e.g., seqFISH) or error correction methods including hybridization chain reaction (HCR) seqFISH and multiplexed error-robust (MER)FISH.

In certain embodiments provided herein are methods for anchoring target nucleic acid within a matrix and clearing non-target cellular components, which can then be used for down stream imaging of the nucleic acid targets. In embodiments, the methods comprise contacting a tissue sample suspected of containing fragments nucleic acid (e.g., formalin fixed paraffin embedded (FFPE) sample) with at least two anchoring agents, wherein the first anchoring agent forms a covalent bond with the target nucleic acid and the second anchoring agent comprises an oligonucleotide that hybridizes with the target nucleic acid; embedding the sample in a polymer matrix wherein the first and second anchoring agents each form a covalent bond with the polymer matrix; and, clearing the non-target cellular components from the polymer matrix wherein the target nucleic acid remains anchored in the polymer matrix to form a matrix anchored target nucleic acid sample. In certain embodiments, the target nucleic acid is RNA, in particular mRNA, wherein the methods comprise contacting a tissue sample suspected of containing fragments RNA (e.g., formalin fixed paraffin embedded (FFPE) tissue sample) with at least two anchoring agents, wherein the first anchoring agent comprises an alkylating agent that forms a covalent bond with the target nucleic acid and the second anchoring agent comprises a polyT sequence that is complementary and hybridizes to the target RNA and wherein the first anchoring agent and the second anchoring agent each comprise an acrydite moiety that covalently binds to the matrix; embedding the sample in a polymer matrix wherein the first and second anchoring agents each form a covalent bond with the polymer matrix; and, clearing the non-target cellular components from the polymer matrix wherein the target RNA remains anchored in the polymer matrix to form a matrix anchored target RNA sample.

In certain embodiments, FFPE tissue sections are prepared for transcriptome analysis (e.g., RNA transcripts) comprising the steps of deparaffinization, ethanol rehydration, antigen retrieval, followed by the addition of at least two anchoring agents (e.g., such as two functionally different or distinct anchoring agents) to the tissue sample, wherein a first anchoring agent forms a covalent bond with the target nucleic acid and a second anchoring agent (also referred to herein as an “anchor probe”) comprises an oligonucleotide that hybridizes with the target nucleic acid. In this way, the target nucleic acid has been functionalized with two separate anchoring agents to form an anchor treated tissue sample. This anchoring treatment step improves immobilization of the target nucleic acid within a gel or polymer matrix, wherein mRNA, in particular, can become fragmented during the FFPE process of fixing tissue sections. Each of these anchoring agents further comprise a chemical moiety (e.g. reactive group) that can form a covalent bond with a polymer matrix either during (polymerization) or after the polymer matrix has formed. Accordingly, after the at least two anchoring agents are added to the tissue sample the sample is then embedded in a polymer matrix wherein the first and second anchoring agents each form a covalent bond with the polymer matrix. In this way, the target nucleic acids are immobilized in the polymer gel matrix. In certain embodiments, the polymer matrix is a polyacrylamide gel and the first and second anchoring agents each comprise a reactive group that will form a covalent bond with acrylamide (e.g. acrydite).

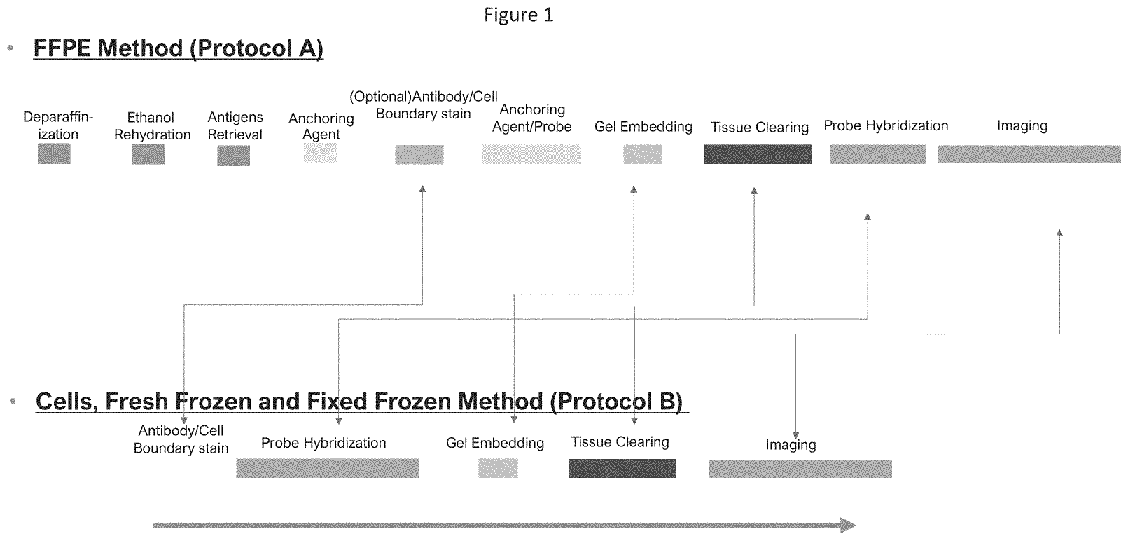

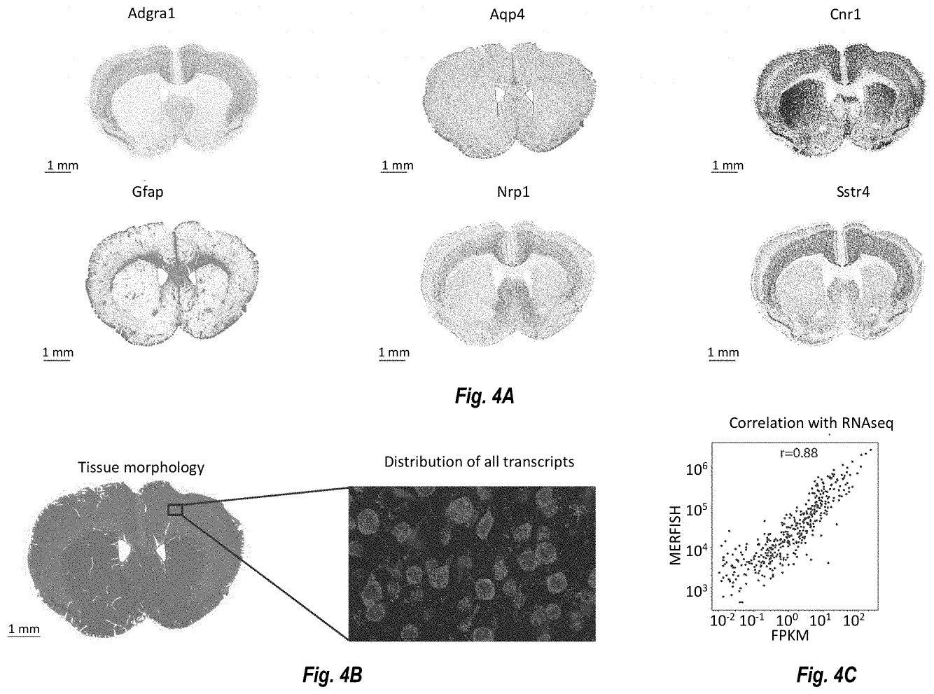

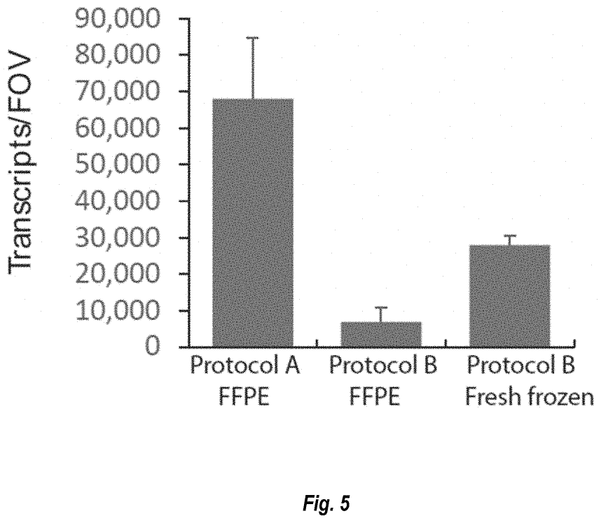

After the gel embedding step, the non-target cellular components (also referred to herein as a tissue clearing or digestion step) are removed using reagents and methods known in the art (e.g. protease digestion). This clearing step, in combination with the use of at least two anchoring agents when starting with FFPE tissue sample, removes the protein crosslinking induced by the formalin fixing process exposing target nucleic acid to a complimentary primary oligonucleotide probe designed to hybridize to a target sequence in the anchored nucleic acid. The comparative Protocol B (See FIG. 1) disclosed herein includes the step of adding a primary oligonucleotide probe before the clearing step, which is adequate for fresh and fixed frozen samples, but significantly reduces the number of imaged transcripts with FFPE samples. See FIG. 4. Applicants have found that use of the combination of two anchoring agents and the order the steps are performed, for example with the primary oligonucleotide probes, added after anchoring and clearing, significantly improves the target nuclei acid available for hybridization and visualization.

Accordingly, following the final step of tissue clearing the original FFPE tissue sample is prepared and ready for transcriptome analysis following protocols known to those skilled in the art (e.g., probe hybridization and imaging). In certain embodiments provided herein are methods for imaging target nucleic acid within a matrix and clearing non-target cellular components comprising: contacting a formalin fixed paraffin embedded (FFPE) tissue sample, or a sample suspected of containing fragmented nucleic acid, with at least two anchoring agents, wherein the first anchoring agent forms a covalent bond with the target nucleic acid and the second anchoring agent comprises an oligonucleotide that hybridizes with the target nucleic acid; embedding the sample in a polymer matrix wherein the first and second anchoring agents each form a covalent bond with the polymer matrix; clearing the non-target cellular components from the polymer matrix wherein the target nucleic acid remains anchored in the polymer matrix to form a matrix anchored target nucleic acid sample; and, contacting the anchored target nucleic acid sample with one or more primary oligonucleotide probes that hybridize to the target nucleic acids and a plurality of secondary nucleic acid probes comprising a fluorescent label and a recognition sequence that hybridizes to a sequence of the primary nucleic acid probe and imaging the target nucleic acids.

In certain embodiments the methods include contacting nucleic acid targets (e.g., RNA transcripts) with at least two anchoring agents to enhance immobilization of RNA transcript efficiency before polymer matrix embedding. In embodiments, the methods include tissue clearing (e.g., removing non-target cellular components) prior to contacting the sample with primary oligonucleotide probes (e.g., MERFISH probes, smFISH probes, etc.) to enhance the efficiency of primary probe binding by exposing target nucleic acid after crosslinked proteins have been cleared. In certain embodiments, the methods comprise contacting the RNA transcripts (e.g., nucleic acid target) with at least two anchoring agents wherein a first anchoring agent forms a covalent bond with the target nucleic acid, and the second anchoring agent (anchor probe) comprises an oligonucleotide that hybridizes with the target nucleic acid, embedding the tissue sample comprising the at least two anchoring agents in a gel matrix whereby the RNA transcripts are immobilized in the polymer gel matrix when the first and second anchoring agents each form a covalent bond with the polymer matrix, digesting or clearing the tissue and/or non-target cellular components followed by contacting the immobilized nucleic acid (e.g., RNA transcripts) with a plurality of primary oligonucleotide probes that specifically hybridize to the immobilized target nucleic acid, and imaging the target nucleic acids.

In certain embodiments provided herein are kits for FFPE sample preparation comprising one or more of the following reagents: deparaffinization buffer, decrosslinking buffer, conditioning buffer, sample prep wash buffer, formamide wash buffer, gel embedding premix, clearing premix, gel coverslip, pre-anchoring activator, anchoring buffer and digestion premix. In certain embodiments, kits of the disclosure comprise at least two “anchoring buffer” formulations; one comprising a first anchoring agent and the other comprising a second anchoring agent or anchoring probe.

An exemplary method, and kit components thereof, according to the present disclosure has been described in Example 1.

Accordingly, in one set of embodiments, a method of detecting (e.g., via imaging) nucleic acid targets in a tissue sample (e.g. FFPE) is provided, the method comprising: contacting a sample containing nucleic acid targets with anchor probes specifically binding (e.g., via hybridization) the nucleic acid targets; immobilizing the nucleic acid target-bound anchor probes in at least part of the sample within a gel (e.g., via covalent attachment of the anchor probe to the polymer gel); clearing the tissue sample within the polymer gel by removing or degrading non-target cellular components (e.g. non-immobilized target nucleic acid); contacting the sample with a plurality of primary nucleic acid probes capable of selectively binding (e.g. hybridizing) the nucleic acid targets; detecting (e.g., via imaging) the nucleic acid probes bound to the nucleic acid targets within the cleared (e.g., gel embedded) sample.

In some embodiments, a method of detecting (e.g., via imaging) nucleic acid targets in a tissue sample (e.g. FFPE) is provided, the method comprising: contacting a tissue sample containing nucleic acid targets with an anchor agent that comprises a first chemical moiety (e.g. a reactive group that can form a covalent bond with the target nucleic acid) that can react with and/or modify an internal base of the nucleic acid targets and a second chemical moiety (e.g., a reactive group that cand form a covalent bond with the polymer gel) that can be incorporated into the polymer gel; immobilizing the nucleic acid target-bound anchor agents in at least part of the tissue sample within a gel (e.g., via covalent attachment of the anchor agent to the polymer gel); clearing the tissue sample within the polymer gel by removing or degrading non-targets (e.g. non-immobilized target nucleic acid); contacting the tissue sample with a plurality of primary nucleic acid probes capable of selectively binding (e.g. hybridizing) the nucleic acid targets; detecting (e.g., via imaging) the nucleic acid probes bound to the nucleic acid targets within the cleared tissue (e.g., gel embedded) sample.

In some embodiments, a method of detecting (e.g., via imaging) nucleic acid targets in a tissue sample (e.g. FFPE) is provided, the method comprising: contacting a tissue sample containing nucleic acid targets with an anchor agent that comprises a first chemical moiety (e.g. a reactive group that can form a covalent bond with the target nucleic acid) that can react with and/or modify an internal base of the nucleic acid targets and a second chemical moiety (e.g., a reactive group that cand form a covalent bond with the polymer gel) that can be incorporated into the polymer gel; contacting the tissue sample with anchor probes specifically binding (e.g., via hybridization) the nucleic acid targets; immobilizing the nucleic acid target-bound anchor probes and the nucleic acid target-bound anchor agents in at least part of the tissue sample within a gel (e.g., via covalent attachment of the anchor agent to the polymer gel); clearing the tissue sample within the polymer gel by removing or degrading non-targets (e.g. non-immobilized target nucleic acid); contacting the cleared tissue sample (e.g., immobilized target nucleic acid sample) with a plurality of primary oligonucleotide nucleic acid probes capable of selectively binding (e.g. hybridizing) the nucleic acid targets; detecting (e.g., via imaging) the nucleic acid probes bound to the immobilized nucleic acid targets within the cleared tissue (e.g., gel embedded) sample.

In certain embodiments, a method for imaging target nucleic acid from formalin-fixed paraffin embedded (FFPE) tissue sample is provided. In embodiments, the method comprises contacting the tissue sample with at least two anchoring agents, wherein the first anchoring agent forms a covalent bond with a target nucleic acid and the second anchoring agent comprises an oligonucleotide that hybridizes with the target nucleic acid; embedding the sample in a polymer gel wherein the first and second anchoring agents each form a covalent bond with the polymer gel; clearing non-immobilized cellular components from the polymer gel to form a gel immobilized target nucleic acid sample; and contacting the immobilized target nucleic acid sample with one or more primary oligonucleotide probes that hybridize to the target nucleic acids, and imaging the target nucleic acids.

In some embodiments, the step of clearing is performed after immobilizing the target nucleic acid in the polymer gel (e.g., after steps of contacting the sample with anchor probes and anchor argents and immobilization the nucleic acid target-bound anchor probes and the nucleic acid target-bound anchor agents) and before the primary oligonucleotide probes are added to the immobilized target nucleic acid. For example, in certain embodiments, a desired target is immobilized within a gel (such as an inert gel matrix), while other components are removed or degraded.

The primary oligonucleotide probes may be, for example, MERFISH probes or smFISH probes, and may be substantially complementary to mRNA or other RNAs, for example, for transcriptome analyses. The primary oligonucleotide probes may also include signaling entities, e.g., fluorescent signaling entities, for imaging and/or analysis of the sample. In certain embodiments, a secondary oligonucleotide probe that hybridizes to the primary oligonucleotide probe comprises an imaging moiety (e.g., fluorescent signaling entities), wherein imaging comprises adding one or more of the secondary probes. In some embodiments, the method further comprises creating codewords or barcodes based on a distribution of the bound nucleic acid probes within the sample. In some embodiments, the method further comprises, for at least some of the codewords, matching the codeword to a valid codeword optionally wherein, if no match is found, applying error correction to the codeword to form a valid codeword or discard the codeword.

### I. TISSUE SAMPLES

As used herein, “tissue sample” herein refers to a collection of similar cells obtained from a tissue of a subject. The tissue may contain nucleated cells with chromosomal material. The source of the tissue sample may be solid tissue, as from a fresh, frozen, FFPE, and/or preserved organ or tissue sample, or biopsy, or aspirate, or blood or any blood constituents, or bodily fluids, such as cerebral spinal fluid, amniotic fluid, peritoneal fluid, or interstitial fluid, or cells from any time in gestation or development of the subject. The tissue sample may also be primary or cultured cells or cell lines, or culture tissues. The tissue sample may contain compounds which are not naturally intermixed with the tissue in nature, such as preservatives, anticoagulants, buffers, fixatives, nutrients, antibiotics, or the like. In some embodiments of the invention, the tissue sample is non-hematologic tissue (i.e., not blood or bone marrow tissue). In embodiments, the tissue sample used in the present methods is a formalin-fixed paraffin embedded tissue sample. While the present methods can be used with any of the tissue samples disclosed herein, the methods provide particular advantages for FFPE tissue samples, or any sample suspected of containing fragmented nucleic acid and/or in accessible nucleic acid targets.

In embodiments, nucleic acid fragmentation can be evaluated and determined using methods well known in the art. For example, to evaluate sample quality for in situ hybridization, it is it is informative to determine the RNA quality of a tissue block using RNA Integrity Number (RIN) or DV200 values via commercially available instruments such as the BioAnalyzer or TapeStation platforms. Briefly, RNA from the samples are extracted first and measured on either BioAnalyzer or TapeStation. RIN is expressed in values that range from 1-10, where 1 indicates a sample has shorter and more degraded RNA whereas 10 reflects longer and less degraded RNA. Higher RIN scores will have more intact 18S and 28S RNAs. DV200 reflects the percentage of RNA fragments greater than 200 nucleotides in length in tissue samples. Tissues with lower DV200 percentages have shorter and more degraded RNA; conversely, a higher percentage indicates longer, and less degraded RNA molecules present in the tissue.

In some embodiments, the tissue sample is a tissue section, a clinical smear, or a cultured cell or tissue. In some embodiments, the tissue sample comprises a tissue section. As used herein, “section” of a tissue sample herein refers to a single part or piece of a tissue sample, for example, a thin slice of tissue or cells cut from a tissue sample. It is understood that multiple sections of tissue samples may be taken and subjected to analysis according to the present invention. In some embodiments, the selected portion or section of tissue comprises a homogeneous population of cells. In some embodiments, the selected portion or section of tissue comprises a heterogeneous population of cells. In some embodiments, the selected portion comprises a region of tissue, e.g., the lumen as a non-limiting example. The selected portion can be as small as one cell or two cells, or could represent many thousands of cells, for example.

Any tissue sample from the subject may be used. Examples of tissue samples that may be used include, but are not limited to, breast, prostate, ovary, colon, lung, endometrium, stomach, salivary gland, or pancreas. The tissue sample can be obtained by a variety of procedures including, but not limited to, surgical excision, aspiration, or biopsy. The tissue may be fresh or frozen.

In some embodiments, the tissue section is a tissue section of brain, adrenal glands, colon, small intestines, stomach, heart, liver, skin, kidney, lung, pancreas, testis, ovary, prostate, uterus, thyroid, and spleen of a mammal (e.g., human or mouse). The methods of the present disclosure may be applied to any type of tissue, including, for example, cancer tissue (including from any cancer). In some embodiments, the tissue section is from a solid tumor. In some embodiments, the tissue sample is from mouse small intestine. In some embodiments, the tissue sample is from mouse brain. In some embodiments, the tissue sample is from human liver cancer. In some embodiments, the tissue sample is from human kidney. In some embodiments, the tissue sample is from human lung. In some embodiments, the tissue sample is from human ovarian cancer. In some embodiments, the tissue sample is from human uterus cancer. In some embodiments, the tissue sample is from human lung cancer.

In some embodiments, the tissue has been stored for a period of time, for example, the period of time that frozen or FFPE are stored. In some embodiments, the tissue sample is a frozen tissue sample. In some embodiments, the tissue is frozen tissue. In some embodiments, the tissue is paraffin-embedded tissue. In some embodiments, the tissue is formalin-fixed paraffin-embedded tissue.

### A. Preparation of Tissue Samples

### 1. Obtaining and Fixing Tissue Samples

Tissue samples can be obtained from an intact organ or tissue using any methods well known to those of skill in the art, e.g., the prior methods used to prepare tissue samples for immunohistochemistry (IHC) or in situ hybridization (ISH) techniques.

For example, any intact organ or tissue may be cut into reasonably small piece(s) (the size of the cut pieces typically ranges from a few millimeters to a few centimeters) and “fixed” to preserve the positions of the nucleic acids within the sample. Techniques for fixing cells and tissues are known to those of ordinary skill in the art. Non-limiting examples of fixatives include such as formaldehyde, paraformaldehyde, glutaraldehyde, ethanol, methanol, acetone, acetic acid, or the like.

In some embodiments, the tissue sample is fixed in a solution containing an aldehyde. In some embodiments, the tissue sample is fixed in a solution containing formalin. In some embodiments, the tissue sample is paraffin embedded. In embodiments, the tissue sample is both formalin-fixed and paraffin-embedded (FFPE).

In addition to intact samples, other samples may be used. In some embodiments, the frozen-sections may be prepared by rehydrating 50 mg of frozen pulverized tissue at room temperature in phosphate-buffered saline (PBS) in a small plastic capsule; pelleting the particles by centrifugation; resuspending the particles in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections. Similarly, permanent tissue sections may be prepared involving rehydration of the 50 mg sample in a plastic microfuge tube; pelleting; resuspending in 10% formalin for a 4 hour fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

In some embodiments, the present invention may utilize standard frozen samples, such as those that are embedded in OCT and that are not pulverized, for example, including those used in standard Frozen Section hospital labs.

Tissue samples are often fixed by conventional methodology. Aldehyde fixatives such as formalin (formaldehyde) and glutaraldehyde are typically used. Tissue samples fixed using other fixation techniques, such as alcohol immersion, are also suitable. See Battifora and Kopinski, J., Histochem. Cytochem., 34:1095 (1986). One of skill in the art will appreciate that the choice of the fixative is determined by the purpose for which the tissue is to be histologically stained or otherwise analyzed. One of skill in the art will also appreciate that the length of fixation depends upon the size of the tissue sample and the fixative used.

The samples used may also be embedded in paraffin. In some embodiments, the tissue sample is fixed and embedded in paraffin or the like. In some embodiments, the tissue sample is both formalin-fixed and paraffin-embedded. In some embodiments, the formalin-fixed paraffin-embedded (FFPE) tissue block is hematoxylin and eosin (H&E) stained. As commonly known in the art, the tissue sample may be first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology. Examples of paraffin that may be used include, but are not limited to, Paraplast, Broloid, and Tissuemay. Once the tissue sample is embedded, the sample may be sectioned by a microtome or the like. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

In some embodiments, the tissue section may range from about 3 μm to about 100 μm, or any intermediate ranges therewithin. In some embodiments, the tissue section may range from about 10 μm to about 100 μm. In some embodiments, the tissue section may range from about m to about 50 μm. In some embodiments, the tissue section may range from about 10 μm to about 30 μm. In some embodiments, the tissue section may range from about 10 μm to about 15 μm. In some embodiments, the tissue section may range from about 3 μm to about 15 μm. In some embodiments, the tissue section may range from about 5 μm to about 20 μm. In some embodiments, the tissue section may range from about 15 μm to about 30 μm. In some embodiments, the tissue section may range about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, or about 20 μm. In some embodiments, the tissue section may range about 30 μm, about 40 μm, about 50 μm, about 60 μm, about 70 μm, about 80 μm, about 90 μm, or about 100 μm.

### 2. Deparaffinization and Rehydration

Tissue sections can be deparaffinized using methods known in the art and/or commercially available kits. The methods remove the bulk of paraffin from the sample. Various techniques are known for deparaffinizing and include, but are not limited to, washing with an organic solvent or agent to dissolve the paraffin.

Exemplar deparaffinization solvents include but are not limited to, benzene, toluene, ethylbenzene, xylenes, D-li-monene, octane, and mixtures thereof. In certain embodiments, the deparaffinization solvents comprise D-limonene. These solvents are preferably of high purity, usually greater than 99%. The volume used and the number of washes necessary will depend on the size of the sample and the amount of paraffin to be removed. A sample may be washed between 1 and about 10 times, or between about two and about four times. A typical volume of organic solvent is about 500 ml for a 10 mm tissue sample.

After deparaffinization, samples may be rehydrated such as by stepwise washing with aqueous lower alcoholic solutions of decreasing concentrations. Ethanol is a preferred lower alcohol for rehydrations while other alcohols may also be used. Non-limiting examples include methanol, isopropanol, and other C1-C5 alcohols. The sample is alternatively vigorously mixed with alcoholic solutions followed by its removal. In some embodiments, deparaffinization and rehydration are carried out simultaneously using a reagent such as EZ-DEWAX™ (BioGenex, San Ramon, Calif.), for example.

In some embodiments, the concentration of alcohol is stepwise lowered. In some embodiments, the concentration range of alcohol is decreased stepwise from about 100% to about 70% in water over about three to five incremental steps. In some embodiments, the concentration range of alcohol is decreased stepwise over three incremental steps with 100%, 90%, and 70% respectively.

### 3. Optional Pretreatment for Antigen Retrieval

In some embodiments of the present disclosure, the samples may be pretreated, such as to facilitate directly or indirectly the methods of the invention. In some embodiments, pretreatment of the tissue increases availability of the target nucleic acid or other targets (e.g., for cell morphology staining). Pretreatments for making targets available (e.g., “antigen retrieval” that retrieves or unmasks the biological markers of interest). An extensive review of antigen retrieval may be found in Shi et al. 1997, J Histochem Cytochem, 45(3):327. Antigen retrieval includes a variety of methods by which the availability of the target for interaction with a specific detection reagent is maximized.

The most common techniques are protease-induced epitope retrieval (PIER) or heat induced epitope retrieval (HIER). Protease-induced epitope retrieval (PIER) may employ enzymes such as proteinase K, pepsin, trypsin, protease, and any subtypes thereof, in an appropriate buffer to restore the epitope for antibody binding. Heat-induced epitope retrieval (HIER) may employ heat to reverse some cross-links and allow the restoration of epitopes. Citrate buffers, Tris, and EDTA base may be employed as exemplary heat-induced reagents in appropriately pH stabilized manner (e.g., 10 mM sodium citrate, 6.0 pH; 1 mM EDTA, pH 8.0; 10 mM Tris base, 1 mM EDTA solution, 0.05% Tween 20, pH 9.0). Detergents (e.g., Tween 20) may be added to the HIER buffer to increase the epitope retrieval. In certain aspects, many proprietary formulations may be available for the PIER or HIER mediate antigen retrieval.

Selective staining may be conducted on a tissue section for detection of biological markers and identification of cell types (e.g., nuclear and/or cell morphology stains). To facilitate the specific recognition of biological markers in fixed tissue (e.g., FFPE tissue sample post-deparaffinization and rehydration), it is often necessary to retrieve or unmask the biological markers of interest, through “antigen retrieval” (also called epitope retrieval or antigen unmasking).

### II. TARGET IMMOBILIZATION

It should be understood that the embedding of the tissue sample within the gel matrix and the immobilization of nucleic acid targets (i.e., covalent attachment of anchor agents to the polymer gel) may be performed in any suitable order in various embodiments as long as they are completed prior to clearing the tissue sample (non-immobilized target nucleic acid) within the gel matrix. It should also be understood that primary probe addition and hybridization happens after the clearing step. In embodiments, the anchoring agents and primary oligonucleotide probes are not added at the same time and/or in the same step. For instance, immobilization of target nucleic acid may occur before, or during embedding of the sample, but the primary oligonucleotide probes are added after the non-target cellular components are removed or cleared from the polymer gel matrix. It is understood that immobilization of the target nucleic acid is a multi-step process, wherein anchoring agents are first added to the tissue sample and a covalent bond is formed between the first anchoring agent and target nucleic acid (as disclosed herein for the first anchor agent of the methods) and the second anchoring agent comprises an oligonucleotide that hybridizes with the target nucleic acid (as disclosed herein for the anchor probe or second anchoring agent of the methods) followed by contact with the polymer matrix wherein both the first and second anchoring agents form covalent bonds with the polymer gel. That entire process immobilizes the target nucleic acid in the polymer gel matrix.

In embodiments, the target nucleic acid-anchoring agents react to form a covalent bond with the polymer gel before, during or after formation of the polymer matrix.

### A. Anchoring Agents

In embodiments, at least two anchoring agents are provided for immobilization of the target nucleic acid (e.g., RNA transcripts) to a polymer matrix, as discussed below. In one embodiment, a first anchoring agent is functionalized to comprise a first chemical moiety or reactive group that will form a covalent bond with the target nucleic acid, and a second chemical moiety or reactive group that will form a covalent bond with the polymer gel matrix. In another embodiment, the second anchoring agent comprises an oligonucleotide that hybridizes with the target nucleic acid and a chemical moiety or reactive group that will form a covalent bond with the polymer gel matrix. In certain embodiments, the chemical moiety or reactive group of the second anchoring agent is the same or different as the second chemical moiety of the first anchoring agent. In certain embodiments, the second anchoring agent is also referred to herein as an anchor probe due to the oligonucleotide that hybridizes to the target nucleic acid. In embodiments, the oligonucleotide portion of the second anchoring agent comprises a poly-T (thymine residues) for hybridizing with the poly-A tail of an mRNA transcript.

In some embodiments, the anchor probes may contain sequences complementary to the desired (target) nucleic acid species, e.g., binding to them via base pairing (hybridizing). In embodiments, anchor probes, comprise a chemical moiety or reactive group able to polymerize (e.g. covalent bonding) with a polymer gel matrix.

In one set of embodiments, the anchoring agents form a covalent bond during the polymerization process with the polymer gel matrix. For example, in the case of polyacrylamide, the anchoring agent may include an acrydite portion that can polymerize and become incorporated into the polymer. In certain embodiments, the second anchoring agent or anchor probe comprises an oligonucleotide (poly-T) that hybridizes with the poly-A tail of mRNA and an acrydite moiety that forms a covalent bond with polyacrylamide, wherein the gel embedding step utilizes polyacrylamide.

The anchoring agents may also contain a portion that can interact with and bind to nucleic acid molecules, or other molecules in which immobilization is desired, e.g., proteins or lipids, other desired targets, etc. The immobilization may be covalent or non-covalent. For example, to immobilize a target nucleic acid, the anchoring agents may comprise a nucleic acid comprising an acrydite portion (e.g., at the 5′ end, the 3′ end, an internal base, etc.) and a nucleic acid sequence substantially complementary to at least a portion of the target nucleic acid. For instance, the nucleic acid may be complementary to at least 5, 6, 7, 8, 9, 10, 11, 12, 13, 14, 15, 16, 17, 18, 19, or 20 or more nucleotides of the nucleic acid. In some cases, the complementarity may be exact (Watson-Crick complementarity), or there may be 1, 2, or more mismatches. In some cases, the anchoring agent can be configured to immobilize mRNA, e.g., in the case of transcriptome analysis. For instance, in one set of embodiments, the anchoring agent may contain a plurality of thymine nucleotides, e.g., sequentially, for binding to the poly-A tail of an mRNA. Thus, for example, the anchoring agent can have at least 5, 6, 7, 8, 9, 10, 11, 12, 13, 14, 15, 16, 17, 18, 19, or 20 or more consecutive thymine nucleotides (e.g., a poly-dT portion) within the anchoring agent. In some cases, at least some of the thymine nucleotides may be “locked” thymine nucleotides. These may comprise at least 20%, at least 30%, at least 40%, at least 50%, at least 60%, at least 70%, or at least 80% of these thymine nucleotides. In certain embodiments, the locked and non-locked nucleotides may alternate. Such locked thymine nucleotides may be useful, for example, to stabilize the hybridization of the poly-A tails of the mRNA with the anchoring agent.

In embodiments, the methods herein further comprise the use of another anchoring agent, referred to herein as a first anchoring agent, wherein that anchoring agent is functionalized with a first and second chemical moiety for covalent attachment to the target nucleic acid, and to the polymer gel matrix. In certain embodiments, the anchoring agent is a derivatized alkylating agent wherein the alkylating agent has been derivatized with a chemical moiety or reactive group that will form a covalent bond with the polymer gel matrix. Alkylating agents are well known in the art, and which form a covalent bond with nucleic acid, including RNA, any of which can be derivatized to form a present anchoring agent. In certain embodiments, the anchoring agent is an alkylating agent derivatized with a reactive group that forms a covalent bond with polyacrylamide. In certain embodiments, the anchoring agent is an alkylating agent that comprises an acrydite moiety. In embodiments, alkylating agents are selected from the group consisting of AltAetamine, Bendamuasrine, Busuilfan, CarlIatin Carmustine, Chlorambucii, Cisplatin, Cyclophosphamide, Dacarbazine, Ifosfamide, Lomustine, Mlechlorethamine, Melphablan, Oxaliplatin, Ternozolomide, Thiotepa and Trabectedin.

In embodiments, the present methods use both an anchoring probe and an anchoring agent (a first and second anchoring agent) for immobilization of the target nucleic acid in the polymer gel matrix. In some embodiments, the nucleic acid targets are immobilized within the gel via both the anchor probes and the anchoring agents bound to the nucleic acid targets.

In one set of embodiments, nucleic acid molecules may be immobilized by covalent bonding. For example, in one set of embodiments, an alkylating agent may be used that covalently binds to nucleic acid molecules and contains a second chemical moiety that can be incorporated into the polyacrylamide as it is polymerized. In yet another set of embodiments, the terminal ribose in an RNA molecule may be oxidized using sodium periodate (or another oxidizing agent) to produce an aldehyde, which may be cross-linked to acrylamide, or other polymer or gel. In other embodiments, chemical agents that are able to modify bases may be used, such as aldehydes, e.g. paraformaldehyde or gluteraldehyde, alkylating agents, or succinimidyl-containing groups; chemical agents that modify the terminal phosphate, such as carboiimides, e.g., EDC (1-ethyl-3-(3-dimethylaminopropyl)carbodiimide); chemical agents that modify internal sugars, such as p-maleimido-phenyl isocyanate; or chemical agents that modify terminal sugars, such as sodium periodate. In some cases, these chemical agents can carry a second chemical moiety that can then be directly cross-linked to the gel or polymer, and/or which can be further modified with a compound that can be directly cross linked to the gel or polymer.

In yet other embodiments, a nucleic acid may be immobilized using anchor probes having substantially complementary portions to the DNA or RNA. There may be 5, 6, 7, 8, 9, 10, 12, 13, 14, 15, 20, 25, 30, 35, 40, 45, 50 or more complementary nucleotides between the anchor probe and the nucleic acid.

### B. Gel Polymer Matrix

In some embodiments, the method disclosed herein further comprises immobilizing the nucleic acid target-bound anchor probes in at least part of the tissue sample within a polymer gel.

The sample may be embedded within a matrix that immobilizes nucleic acid targets. For instance, the matrix may comprise a gel or a polymer, such as polyacrylamide. Thus, for example, acrylamide and a suitable cross-linker (e.g., N,N′-methylenebisacrylamide) can be added to the sample and polymerized to form a gel. The anchor probes, if present, may include a portion able to polymerize with the gel (e.g., an acrydite moiety) during the polymerization process, and nucleic acids (e.g., mRNAs containing poly-A tails) may then be able to associate with the anchor portion. In such fashion, the mRNAs may be immobilized to the polyacrylamide gel. As another example, DNA and/or RNA molecules may be immobilized to the polyacrylamide gel using anchor probes having substantially complementary portions to the DNA or RNA. As yet another example, DNA and/or RNA molecules may be physically tangled within the polyacrylamide gel, e.g., due to their length, to immobilize them to the polyacrylamide gel.

The sample may be immobilized or embedded within a polymer or a gel, partially or completely. In some cases, the sample may be embedded within a relatively large polymer or gel, which can then be sectioned or sliced in some cases to produce smaller portions for analysis, e.g., using various microtomy techniques commonly available to those of ordinary skill in the art. For instance, tissues or organs may be immobilized within a suitable polymer or gel.

A variety of polymers may be used in some embodiments. In some cases, the polymer may be selected to be relatively optically transparent. The polymer may also be one that does not significantly distort during the polymerization process, although in some cases, the polymer may exhibit some distortion. In some cases, the amount of distortion may be determined as a relative change in size that is less than 5, less than 4, less than 3, less than 2, less than 1.5, less than 1.3, or less than 1.2 (i.e., a change in size of 2 means that a sample doubles in linear dimension), or inverses of these (i.e., an inverse change in size of 2 means that a sample halves in linear dimensions).

Examples of suitable polymers include polyacrylamide and agarose. In embodiments, the polymer is not a hydrogel and/or does not comprise polymers or monomers that swell or expand. A variety of polymers could be used in various embodiments that involve chemical cross links between gel subunits, including but not limited to acrylic acid, acrylamide, ethylene glycol diacrylate, ethylene glycol dimetharcrylate, poly(ethylene glycol dimethacrylate); and/or hydrophobic or hydrogen bonding interactions, such as poly(N-isopropyl acrylamide), methyl cellulose, (ethylene oxide)-(propylene oxide)-(ethylene oxide terpolymers, sodium alginate, poly(vinyl alcohol), alignate, chitosan, gum Arabic, gelatin, and agarose.

### III. TISSUE CLEARING

After immobilization of nucleic acids targets to the gel, other components that are not the desired targets (e.g., non-immobilized target nucleic acid) within the sample may be removed or degraded.

By “clearing” a tissue sample or a “cleared” tissue sample, it is meant that the tissue sample is made substantially permeable to light, i.e., transparent, and the optical properties of the sample change to allow more light to pass through the sample. In some embodiments, about 70% or more of the light (e.g., white light, ultraviolet light or infrared light) that is used to illuminate the sample will pass through the sample and illuminate only selected cellular components (e.g., nucleic acids) therein, e.g., 75% or more of the light, 80% or more of the light, 85% or more of the light, 90% or more of the light, 95% or more of the light, 98% or more of the light, e.g. 100% of the light will pass through the specimen. Any treatment known for tissue clearing may be used to clear the tissue sample in the methods described herein, which are further discussed below.

Details of tissue clearing have been further discussed in US Patent Publ. No. 2019/0264270 published Aug. 29, 2019, entitled “Matrix imprinting and clearing,” the content of which is incorporated herein by reference in its entirety. Such clearance may include removal (e.g., physical removal) of cellular components from the sample, and/or degradation within the sample, such that they are no longer as prominent within the background. Degradation may include, for example, chemical degradation, enzymatic degradation, or the like.

In some cases, at least 50%, at least 60%, at least 70%, at least 80%, or at least 90% of the undesired components within the sample may be removed or degraded. Such clearance may include physical removal or degradation of the components (e.g., to smaller components, components that are not fluorescent, etc.). Removal or degradation of such components may decrease background fluorescence or autofluorescence within the sample during analysis.

Multiple clearance steps can also be performed in certain embodiments, e.g., to remove or degrade various undesired components.

For example, enzymes, denaturants, chelating agents, chemical agents, and the like, may break down the proteins into smaller components and/or amino acids. These smaller components may be easier to remove physically, and/or may be sufficiently small or inert such that they do not significantly affect the background. Similarly, lipids may be removed or degraded from the sample using surfactants or the like. In some cases, one or more of these are used, e.g., simultaneously or sequentially. Non-limiting examples of suitable enzymes include proteinases such as proteinase K, proteases or peptidases, or digestive enzymes such as trypsin, pepsin, or chymotrypsin. Non-limiting examples of suitable denaturants include guanidine HCl, acetone, acetic acid, urea, or lithium perchlorate. Non-limiting examples of chemical agents able to denature proteins include solvents such as phenol, chloroform, guanidinium isocyananate, urea, formamide, etc. Non-limiting examples of surfactants include Triton X-100 (polyethylene glycol p-(1,1,3,3-tetramethylbutyl)-phenyl ether), SDS (sodium dodecyl sulfate), Igepal CA-630, or poloxamers. Non-limiting examples of chelating agents include ethylenediaminetetraacetic acid (EDTA), citrate, or polyaspartic acid. In some embodiments, compounds such as these may be applied to the sample to remove or degrade proteins, lipids, and/or other components. For instance, a buffer solution (e.g., containing Tris or tris(hydroxymethyl)aminomethane) may be applied to the sample, then removed.

Non-limiting examples of techniques to remove or degrade RNA include RNA enzymes such as Rnase A, Rnase T, or Rnase H, or chemical agents, e.g., via alkaline hydrolysis (for example, by increasing the pH to greater than 10). Non-limiting examples of systems to remove or degrade sugars or extracellular matrix include enzymes such as chitinase, heparinases, or other glycosylases. Non-limiting examples of systems to remove or degrade lipids include enzymes such as lipidases, chemical agents such as alcohols (e.g., methanol or ethanol), or detergents such as Triton X-100 or sodium dodecyl sulfate. Many of these are readily available commercially. In this way, the background of the sample may be reduced, which may facilitate analysis of the nucleic acid probes or other desired targets, e.g., using fluorescence microscopy, or other techniques as discussed herein.

### IV. FORMATION OF NUCLEIC ACID TARGET/PROBE COMPLEX

### A. Nucleic Acid Targets

The nucleic acid targets may be, for example, DNA, RNA, or other nucleic acids that are present in a cell within a tissue sample.

In some embodiments, the nucleic acid target is RNA. The RNA may be coding and/or non-coding RNA. Non-limiting examples of RNA that may be studied within the cell include mRNA, siRNA, rRNA, miRNA, tRNA, lncRNA, snoRNAs, snRNAs, exRNAs, piRNAs, or the like.

The nucleic acids may be endogenous to the cell, or added to the cell. For instance, the nucleic acid may be viral, or artificially created. In some cases, the nucleic acid to be determined may be expressed by the cell.

In some cases, a significant portion of the nucleic acid within the cell may be studied. For instance, in some cases, enough of the RNA present within a cell may be determined so as to produce a partial or complete transcriptome of the cell. In some cases, at least 4 unique mRNA gene transcripts are determined within a cell, and in some cases, at least 3, at least 4, at least 7, at least 8, at least 12, at least 14, at least 15, at least 16, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 types of mRNAs may be determined within a cell.

In some cases, the transcriptome of a cell may be determined. It should be understood that the transcriptome generally encompasses all RNA transcript molecules produced within a cell, coding and non-coding not just coding messenger RNA. Thus, for instance, the transcriptome may also include non-coding rRNA, tRNA, siRNA, miRNA, etc. In some embodiments, at least 5%, at least 10%, at least 15%, at least 20%, at least 25%, at least 30%, at least 40%, at least 50%, at least 60%, at least 70%, at least 80%, at least 90%, or 100% of the transcriptome of a cell may be determined.

The determination of one or more nucleic acids within the sample may be qualitative and/or quantitative. In addition, the determination may also be spatial, e.g., the position of the nucleic acid within the sample may be determined in two or three dimensions. In some embodiments, the positions, number, and/or concentrations of nucleic acids within the cell (or other sample) may be determined.

### B. Nucleic Acid Probes

In one set of embodiments, as an illustrative non-limiting example, the non-target cellular component cleared and immobilized target nucleic acid sample (“matrix anchored target nucleic acid sample”) may be studied by exposing it to one or more types of primary oligonucleotide nucleic acid probes, with may be imaged using secondary nucleic acid probes (e.g., fluorescent labeled) either simultaneously or sequentially.

For instance, in one set of embodiments, the nucleic acid probes may include smFISH or MERFISH probes, such as those discussed in U.S. Pat. No. 11,098,303 or U.S. Pat. No. 10,240,146, each incorporated herein by reference in its entirety.

The nucleic acid probes may comprise nucleic acids (or entities that can hybridize to a nucleic acid, e.g., specifically) such as DNA, RNA, LNA (locked nucleic acids), PNA (peptide nucleic acids), or combinations thereof. In some cases, additional components may also be present within the nucleic acid probes, e.g., as discussed below. Any suitable method may be used to introduce nucleic acid probes into a sample.

The nucleic acid probes are added to the sample comprising the gel immobilized target nucleic acid after the non-target cellular components have been removed from the gel. Certain aspects of the present invention are generally directed to nucleic acid probes that are introduced into a sample. The probes may comprise any of a variety of entities that can hybridize to a nucleic acid, typically by Watson-Crick base pairing, such as DNA, RNA, LNA, PNA, etc., depending on the application. The nucleic acid probe typically contains a target sequence that is able to bind to at least a portion of a target nucleic acid, in some cases specifically. When introduced into a sample, the nucleic acid probe may be able to bind to a specific target nucleic acid (e.g., an mRNA, or other nucleic acids as discussed herein). In some cases, the nucleic acid probes may be determined using signaling entities (e.g., as discussed below), and/or by using secondary nucleic acid probes able to bind to the nucleic acid probes (i.e., to primary nucleic acid probes). The determination of such nucleic acid probes is discussed in detail below.

In some cases, more than one distinct (primary) nucleic acid probe may be applied to a sample, e.g., simultaneously. For example, there may be at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable nucleic acid probes that are applied to a sample, e.g., simultaneously or sequentially.

In certain embodiments, the primary oligonucleotide probes comprise a target sequence designed to hybridize with the anchored target nucleic acid. The target sequence may be positioned anywhere within the nucleic acid probe (or primary nucleic acid probe or encoding nucleic acid probe). The target sequence may contain a region that is substantially complementary to a portion of a target nucleic acid. In some cases, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary. In some cases, the target sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the target sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the target sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc. Typically, complementarity is determined on the basis of Watson-Crick nucleotide base pairing.

The target sequence of a (primary) nucleic acid probe may be determined with reference to a target nucleic acid suspected of being present within a sample. For example, a target nucleic acid to a protein may be determined using the protein's sequence, by determining the nucleic acids that are expressed to form the protein. In some cases, only a portion of the nucleic acids encoding the protein are used, e.g., having the lengths as discussed above. In addition, in some cases, more than one target sequence that can be used to identify a particular target may be used. For instance, multiple probes can be used, sequentially and/or simultaneously, that can bind to or hybridize to different regions of the same target. Hybridization typically refers to an annealing process by which complementary single-stranded nucleic acids associate through Watson-Crick nucleotide base pairing (e.g., hydrogen bonding, guanine-cytosine and adenine-thymine) to form double-stranded nucleic acid.

In some embodiments, a nucleic acid probe, such as a primary nucleic acid probe, may also comprise one or more “read” sequences designed to hybridize with secondary nucleic acid probes comprising a label (e.g. fluorescent label). However, it should be understood that read sequences are not necessary in all cases. In some embodiments, the nucleic acid probe may comprise 1, 2, 3, 4, 5, 6, 7, 8, 9, 10, 11, 12, 13, 14, 15, 16 or more, 20 or more, 32 or more, 40 or more, 50 or more, 64 or more, 75 or more, 100 or more, 128 or more read sequences. The read sequences may be positioned anywhere within the nucleic acid probe. If more than one read sequence is present, the read sequences may be positioned next to each other, and/or interspersed with other sequences. In certain embodiments, the primary oligonucleotide probes comprise one read sequence. In certain other embodiments, the primary oligonucleotide probes comprise two read sequences, which may the same or distinct from each other (e.g., meaning a secondary nucleic acid probe will not hybridize to distinct read sequences).

The read sequences, if present, may be of any length. If more than one read sequence is used, the read sequences may independently have the same or different lengths. For instance, the read sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the read sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the read sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

The read sequence may be arbitrary or random in some embodiments. In certain cases, the read sequences are chosen so as to reduce or minimize homology with other components of the sample, e.g., such that the read sequences do not themselves bind to or hybridize with other nucleic acids suspected of being within the sample. In some cases, the homology may be less than 10%, less than 8%, less than 7%, less than 6%, less than 5%, less than 4%, less than 3%, less than 2%, or less than 1%. In some cases, there may be a homology of less than 20 base pairs, less than 18 base pairs, less than 15 base pairs, less than 14 base pairs, less than 13 base pairs, less than 12 base pairs, less than 11 base pairs, or less than 10 base pairs. In some cases, the base pairs are sequential.

In certain embodiments, primary oligonucleotide probes are provided as a pool of probes, wherein each pool of nucleic acid probes hybridize to a distinct target nucleic sequence (e.g., distinct RNA transcript). In embodiments each pool of probes encode, via read sequences, a N-bit binary code that was assigned to each distinct RNA transcript. In certain embodiments, the N-bit binary code has a Hamming weight of at least 2, at least 4, at least 5, at least 6, at least 7 or at least 8, wherein the Hamming weight value is the number of “1” values in the N-bit code and all other positions are “0”. In embodiments the N-bit binary code has a Hamming weight of at least 2, or at least 4, meaning the code contains two or four “1” bit values, respectively, and the other bit positions are “0”. In embodiments, the N-bit binary code has an N value of 3 to 100, with any value thereof possible. In certain embodiments, the binary code is a 4-bit binary code, a 6-bit binary code, a 8-bit binary code, a 16-bit binary code, a 36-bit binary code, a 50-bit binary code, a 54-bit binary code or a 100-bit binary code, or any combination thereof. Each position of the binary code is either a “0” or a “1”, wherein the binding of secondary probes to the read sequence determines if the hybridization read is “0”, wherein no probe binds, or a “1” wherein secondary probe bound to the read sequence of the primary probe. Sequential hybridization and imaging of the secondary read out probes is performed until each position of the N-bit binary code has been read providing a barcode or codeword for the target nucleic acid (e.g. mRNA sequence).

In one set of embodiments, a population of nucleic acid probes may contain a certain number of read sequences, which may be less than the number of targets of the nucleic acid probes in some cases. Those of ordinary skill in the art will be aware that if there is one signaling entity and n read sequences, then in general 2n−1 different nucleic acid targets may be uniquely identified. However, not all possible combinations need be used. For instance, a population of nucleic acid probes may target 12 different nucleic acid sequences, yet contain no more than 8 read sequences. As another example, a population of nucleic acids may target 140 different nucleic acid species, yet contain no more than 16 read sequences. Different nucleic acid sequence targets may be separately identified by using different combinations of read sequences within each probe. For instance, each probe may contain 1, 2, 3, 4, 5, 6, 7, 8, 9, 10, 11, 12, 13, 14, 15, etc. or more read sequences. In some cases, a population of nucleic acid probes may each contain the same number of read sequences, although in other cases, there may be different numbers of read sequences present on the various probes.

As a non-limiting example, a first nucleic acid probe may contain a first target sequence, a first read sequence, and a second read sequence, while a second, different nucleic acid probe may contain a second target sequence, the same first read sequence, but a third read sequence instead of the second read sequence. Such probes may thereby be distinguished by determining the various read sequences present or associated with a given probe or location, as discussed herein.

In addition, the nucleic acid probes (and their corresponding, complimentary sites on the encoding probes), in certain embodiments, may be made using only 2 or only 3 of the 4 bases, such as leaving out all the Gs or leaving out all of the Cs within the probe. Sequences lacking either Gs or Cs may form very little secondary structure in certain embodiments, and can contribute to more uniform, faster hybridization.

In some embodiments, the nucleic acid probe may contain a signaling entity. It should be understood that signaling entities are not required in all cases, however; for instance, the nucleic acid probe may be determined using secondary nucleic acid probes in some embodiments, as is discussed in additional detail below. Examples of signaling entities that can be used are also discussed in more detail below.

Other components may also be present within a nucleic acid probe as well. For example, in one set of embodiments, one or more primer sequences may be present, e.g., to allow for enzymatic amplification of probes. Those of ordinary skill in the art will be aware of primer sequences suitable for applications such as amplification (e.g., using PCR or other suitable techniques). Many such primer sequences are available commercially. Other examples of sequences that may be present within a primary nucleic acid probe include, but are not limited to promoter sequences, operons, identification sequences, nonsense sequences, or the like.

Typically, a primer is a single-stranded or partially double-stranded nucleic acid (e.g., DNA) that serves as a starting point for nucleic acid synthesis, allowing polymerase enzymes such as nucleic acid polymerase to extend the primer and replicate the complementary strand. A primer is (e.g., is designed to be) complementary to and to hybridize to a target nucleic acid. In some embodiments, a primer is a synthetic primer. In some embodiments, a primer is a non-naturally occurring primer. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides.

In addition, the components of the nucleic acid probe may be arranged in any suitable order. For instance, in one embodiment, the components may be arranged in a nucleic acid probe as: primer-read sequences-targeting sequence-read sequences-reverse primer. The “read sequences” in this structure may each contain any number (including 0) of read sequences, so long as at least one read sequence is present in the probe. Non-limiting example structures include:
primer-targeting sequence-read sequences-reverse primer,
primer-read sequences-targeting sequence-reverse primer,
targeting sequence-primer-targeting sequence-read sequences-reverse primer,
targeting sequence-primer-read sequences-targeting sequence-reverse primer,
primer-target sequence-read sequences-targeting sequence-reverse primer,
targeting sequence-primer-read sequence-reverse primer,
targeting sequence-read sequence-primer,
read sequence-targeting sequence-primer,
read sequence-primer-targeting sequence-reverse primer, etc.
   In addition, the reverse primer is optional in some embodiments, including in all of the above-described examples.

### V. DETECTION/IMAGING OF NUCLEIC ACID TARGET/PROBE COMPLEX

After introduction of the primary nucleic acid probes into a sample, the nucleic acid probes may be directly determined by determining signaling entities (if present), and/or the nucleic acid probes may be determined by using one or more secondary nucleic acid probes (also referred to herein as read out probes), in accordance with certain aspects of the invention. As mentioned, in some cases, the determination may be spatial, e.g., in two or three dimensions. In addition, in some cases, the determination may be quantitative, e.g., the amount or concentration of a primary nucleic acid probe (and of a target nucleic acid) may be determined. Additionally, the secondary probes may comprise any of a variety of entities able to hybridize a nucleic acid, e.g., DNA, RNA, LNA, and/or PNA, etc., depending on the application. Signaling entities are discussed in more detail below.

A secondary nucleic acid probe may contain a recognition sequence able to bind to or hybridize with a read sequence of a primary nucleic acid probe. In some cases, the binding is specific, or the binding may be such that a recognition sequence preferentially binds to or hybridizes with only one of the read sequences that are present. The secondary nucleic acid probe may also contain one or more signaling entities. If more than one secondary nucleic acid probe is used, the signaling entities may be the same or different. In embodiments, the secondary nucleic acid probe comprises a fluorescent label and may be referred to herein as a fluorescent secondary nucleic acid probe.

The recognition sequences may be of any length, and multiple recognition sequences may be of the same or different lengths. If more than one recognition sequence is used, the recognition sequences may independently have the same or different lengths. For instance, the recognition sequence may be at least 5, at least 10, at least 15, at least 20, at least 25, at least 30, at least 35, at least 40, or at least 50 nucleotides in length. In some cases, the recognition sequence may be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the recognition sequence may have a length of between 10 and 30, between 20 and 40, or between 25 and 35 nucleotides, etc. In one embodiment, the recognition sequence is of the same length as the read sequence. In addition, in some cases, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to a read sequence of the primary nucleic acid probe.

As mentioned, in some cases, the secondary nucleic acid probe may comprise one or more signaling entities. Examples of signaling entities are discussed in more detail below.

As discussed, in certain aspects of the invention, nucleic acid probes are used that contain various “read sequences.” For example, a population or pool of primary nucleic acid probes may contain certain “read sequences” which can bind certain of the secondary nucleic acid probes, and the locations of the primary nucleic acid probes are determined within the sample using secondary nucleic acid probes, e.g., which comprise a signaling entity. As mentioned, in some cases, a population of read sequences may be combined in various combinations to produce different nucleic acid probes, e.g., such that a relatively small number of read sequences may be used to produce a relatively large number of different nucleic acid probes.

Thus, in some cases, a population of primary nucleic acid probes (or other nucleic acid probes) may each contain a certain number of read sequences, some of which are shared between different primary nucleic acid probes such that the total population of primary nucleic acid probes may contain a certain number of read sequences. A population of nucleic acid probes may have any suitable number of read sequences. For example, a population of primary nucleic acid probes may have 1, 2, 3, 4, 5, 6, 7, 8, 9, 10, 11, 12, 13, 14, 15, 16, 17, 18, 19, 20 etc. read sequences. More than 20 are also possible in some embodiments. In addition, in some cases, a population of nucleic acid probes may, in total, have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 20 or more, 24 or more, 32 or more, 40 or more, 50 or more, 60 or more, 64 or more, 100 or more, 128 or more, etc. of possible read sequences present, although some or all of the probes may each contain more than one read sequence, as discussed herein. In addition, in some embodiments, the population of nucleic acid probes may have no more than 100, no more than 80, no more than 64, no more than 60, no more than 50, no more than 40, no more than 32, no more than 24, no more than 20, no more than 16, no more than 15, no more than 14, no more than 13, no more than 12, no more than 11, no more than 10, no more than 9, no more than 8, no more than 7, no more than 6, no more than 5, no more than 4, no more than 3, or no more than two read sequences present. Combinations of any of these are also possible, e.g., a population of nucleic acid probes may comprise between 10 and 15 read sequences in total.

As a non-limiting example of an approach to combinatorially producing a relatively large number of nucleic acid probes from a relatively small number of read sequences, in a population of 6 different types of nucleic acid probes, each comprising one or more read sequences, the total number of read sequences within the population may be no greater than 4. It should be understood that although 4 read sequences are used in this example for ease of explanation, in other embodiments, larger numbers of nucleic acid probes may be realized, for example, using 5, 8, 10, 16, 32, etc. or more read sequences, or any other suitable number of read sequences described herein, depending on the application. If each of the primary nucleic acid probes contains two different read sequences, then by using 4 such read sequences (A, B, C, and D), up to 6 probes may be separately identified. It should be noted that in this example, the ordering of read sequences on a nucleic acid probe is not essential, i.e., “AB” and “BA” may be treated as being synonymous (although in other embodiments, the ordering of read sequences may be essential and “AB” and “BA” may not necessarily be synonymous). Similarly, if 5 read sequences are used (A, B, C, D, and E) in the population of primary nucleic acid probes, up to 10 probes may be separately identified. For example, one of ordinary skill in the art would understand that, for k read sequences in a population with n read sequences on each probe, up to x different probes may be produced, assuming that the ordering of read sequences is not essential; because not all of the probes need to have the same number of read sequences and not all combinations of read sequences need to be used in every embodiment, either more or less than this number of different probes may also be used in certain embodiments. In addition, it should also be understood that the number of read sequences on each probe need not be identical in some embodiments. For instance, some probes may contain 2 read sequences while other probes may contain 3 read sequences.

In some aspects, the read sequences and/or the pattern of binding of nucleic acid probes within a sample may be used to define an error-detecting and/or an error-correcting code, for example, to reduce or prevent misidentification or errors of the nucleic acids. Thus, for example, if binding is indicated (e.g., as determined using a signaling entity), then the location may be identified with a “1”; conversely, if no binding is indicated, then the location may be identified with a “0” (or vice versa, in some cases), when using a pool of primary nucleic acid probes comprising read sequences, wherein each pool each pool of probes encode, via read sequences, a N-bit binary code with a Hamming weight of at least 2 that was assigned to each distinct target nucleic acid (e.g. RNA transcript). Multiple rounds of binding determinations, e.g., using different nucleic acid probes, can then be used to create a “codeword,” e.g., for that spatial location based on binding of the read out probes to the read sequences of the primary probes. In some embodiments, the N-bit binary code may be subjected to error detection and/or correction. For instance, the codewords may be organized such that, if no match is found for a given set of read sequences or binding pattern of nucleic acid probes, then the match may be identified as an error, and optionally, error correction may be applied sequences to determine the correct target for the nucleic acid probes. In some cases, the codewords may have fewer “letters” or positions that the total number of nucleic acids encoded by the codewords, e.g. where each codeword encodes a different nucleic acid.

Such error-detecting and/or the error-correction code may take a variety of forms. A variety of such codes have previously been developed in other contexts such as the telecommunications industry, such as Golay codes or Hamming codes. In one set of embodiments, the read sequences or binding patterns of the nucleic acid probes are assigned such that not every possible combination is assigned.

For example, if 4 read sequences are possible and a primary nucleic acid probe contains 2 read sequences, then up to 6 primary nucleic acid probes could be identified; but the number of primary nucleic acid probes used may be less than 6. Similarly, for k read sequences in a population with n read sequences on each primary nucleic acid probe, different probes may be produced, but the number of primary nucleic acid probes that are used may be any number more or less than k. In addition, these may be randomly assigned, or assigned in specific ways to increase the ability to detect and/or correct errors.

As another example, if multiple rounds of nucleic acid probes are used, the number of rounds may be arbitrarily chosen. If in each round, each target can give two possible outcomes, such as being detected or not being detected, up to 2n different targets may be possible for n rounds of probes, but the number of nucleic acid targets that are actually used may be any number less than 2n. For example, if in each round, each target can give more than two possible outcomes, such as being detected in different color channels, more than 2n (e.g. 3n, 4n . . . ) different targets may be possible for n rounds of probes. In some cases, the number of nucleic acid targets that are actually used may be any number less than this number. In addition, these may be randomly assigned, or assigned in specific ways to increase the ability to detect and/or correct errors.

For example, in one set of embodiments, the codewords or nucleic acid probes may be assigned within a code space such that the assignments are separated by a Hamming distance, which measures the number of incorrect “reads” in a given pattern that cause the nucleic acid probe to be misinterpreted as a different valid nucleic acid probe. The Hamming weight refers to the distance between the N-bit binary code assigned to a target and thus each pool of primary oligonucleotide probes as encoded via their read sequences. In embodiments a pool of primary probes may have an assigned N-bit binary code with a Hamming weight of at least 4 and a Hamming weight between pools of 4. In that embodiment, errors can both be detected and corrected. In certain cases, the Hamming distance may be at least 2, at least 3, at least 4, at least 5, at least 6, or the like. In addition, in one set of embodiments, the assignments may be formed as a Hamming code, for instance, a Hamming(7, 4) code, a Hamming(15, 11) code, a Hamming(31, 26) code, a Hamming(63, 57) code, a Hamming(127, 120) code, etc. In another set of embodiments, the assignments may form a SECDED code, e.g., a SECDED(8,4) code, a SECDED(16,4) code, a SCEDED(16, 11) code, a SCEDED(22, 16) code, a SCEDED(39, 32) code, a SCEDED(72, 64) code, etc. In yet another set of embodiments, the assignments may form an extended binary Golay code, a perfect binary Golay code, or a ternary Golay code. In another set of embodiments, the assignments may represent a subset of the possible values taken from any of the codes described above.

For example, a code with the same error correcting properties of the SECDED code may be formed by using only binary words that contain a fixed number of ‘1’ bits, such as 4, to encode the targets. In another set of embodiments, the assignments may represent a subset of the possible values taken from codes described above for the purpose of addressing asymmetric readout errors. For example, in some cases, a code in which the number of ‘1’ bits may be fixed for all used binary words may eliminate the biased measurement of words with different numbers of ‘1’s when the rate at which ‘0’ bits are measured as ‘1’s or ‘1’ bits are measured as ‘0’s are different.

Accordingly, in some embodiments, once the codeword is determined (e.g., as discussed herein), the codeword may be compared to the known nucleic acid codewords. If a match is found, then the nucleic acid target can be identified or determined. If no match is found, then an error in the reading of the codeword may be identified. In some cases, error correction can also be applied to determine the correct codeword, and thus resulting in the correct identity of the nucleic acid target. In some cases, the codewords may be selected such that, assuming that there is only one error present, only one possible correct codeword is available, and thus, only one correct identity of the nucleic acid target is possible. In some cases, this may also be generalized to larger codeword spacings or Hamming distances; for instance, the codewords may be selected such that if two, three, or four errors are present (or more in some cases), only one possible correct codeword is available, and thus, only one correct identity of the nucleic acid targets is possible.

The error-correcting code may be a binary error-correcting code, or it may be based on other numbering systems, e.g., ternary or quaternary error-correcting codes. For instance, in one set of embodiments, more than one type of signaling entity may be used and assigned to different numbers within the error-correcting code. Thus, as a non-limiting example, a first signaling entity (or more than one signaling entity, in some cases) may be assigned as “1” and a second signaling entity (or more than one signaling entity, in some cases) may be assigned as “2” (with “0” indicating no signaling entity present), and the codewords distributed to define a ternary error-correcting code. Similarly, a third signaling entity may additionally be assigned as “3” to make a quaternary error-correcting code, etc.

The contents of each of the following references are incorporated herein by reference: U.S. Pat. No. 11,098,303, entitled “Systems and Methods for Determining Nucleic Acids”; U.S. Pat. No. 10,240,146, entitled “Probe Library Construction”; US Patent Publ. No. 2019-0264270, entitled “Matrix Imprinting and Clearing”; and US Patent Publ. No. 2022-0064697, entitled “Amplification methods and systems for MERFISH and other applications,” for further discussions of Multiplexed Error-Robust Fluorescence In Situ Hybridization (MERFISH) and its examples (e.g., MERFISH probes described herein, signal amplification, determining nucleic acid probes, creating codewords, and error detection and correction, etc.).

As discussed above, in certain aspects, signaling entities are determined, e.g., to determine nucleic acid probes and/or to create codewords. In some cases, signaling entities within a sample may be determined, e.g., spatially, using a variety of techniques. In some embodiments, the signaling entities may be fluorescent, and techniques for determining fluorescence within a sample, such as fluorescence microscopy or confocal microscopy, may be used to spatially identify the positions of signaling entities within a cell. In some cases, the positions of entities within the sample may be determined in two or even three dimensions. In addition, in some embodiments, more than one signaling entity may be determined at a time (e.g., signaling entities with different colors or emissions), and/or sequentially.

In addition, in some embodiments, a confidence level for the identified nucleic acid target may be determined. For example, the confidence level may be determined using a ratio of the number of exact matches to the number of matches having one or more one-bit errors. In some cases, only matches having a confidence ratio greater than a certain value may be used. For instance, in certain embodiments, matches may be accepted only if the confidence ratio for the match is greater than about 0.01, greater than about 0.03, greater than about 0.05, greater than about 0.1, greater than about 0.3, greater than about 0.5, greater than about 1, greater than about 3, greater than about 5, greater than about 10, greater than about 30, greater than about 50, greater than about 100, greater than about 300, greater than about 500, greater than about 1000, or any other suitable value. In addition, in some embodiments, matches may be accepted only if the confidence ratio for the identified nucleic acid target is greater than an internal standard or false positive control by about 0.01, about 0.03, about 0.05, about 0.1, about 0.3, about 0.5, about 1, about 3, about 5, about 10, about 30, about 50, about 100, about 300, about 500, about 1000, or any other suitable value

In some embodiments, the spatial positions of the entities (and thus, nucleic acid probes that the entities may be associated with) may be determined at relatively high resolutions. For instance, the positions may be determined at spatial resolutions of better than about 100 micrometers, better than about 30 micrometers, better than about 10 micrometers, better than about 3 micrometers, better than about 1 micrometer, better than about 800 nm, better than about 600 nm, better than about 500 nm, better than about 400 nm, better than about 300 nm, better than about 200 nm, better than about 100 nm, better than about 90 nm, better than about 80 nm, better than about 70 nm, better than about 60 nm, better than about 50 nm, better than about 40 nm, better than about 30 nm, better than about 20 nm, or better than about 10 nm, etc.

There are a variety of techniques able to determine or image the spatial positions of entities or targets optically, e.g., using fluorescence microscopy, using radioactivity, via conjugation with suitable chromophores, or the like. For example, various conventional microscopy techniques that may be used in various embodiments of the invention include, but are not limited to, epi-fluorescence microscopy, total-internal-reflectance microscopy, highly inclined thin-illumination (HILO) microscopy, light-sheet microscopy, scanning confocal microscopy, scanning line confocal microscopy, spinning disk confocal microscopy, or other comparable conventional microscopy techniques.

In some embodiments, in situ hybridization (ISH) techniques for labeling nucleic acids such as DNA or RNA may be used, e.g., where nucleic acid probes may be hybridized to nucleic acids in samples. These may be performed, e.g., at cellular-scale or single-molecule-scale resolutions. In some cases, the ISH probes can be composed of RNA, DNA, PNA, LNA, other synthetic nucleotides, or the like, and/or a combination of any of these. The presence of a hybridized probe can be measured, for example, with radioactivity using radioactively labeled nucleic acid probes, immunohistochemistry using, for example, biotin labeled nucleic acid probes, enzymatic chromophore or fluorophore generation using, for example, probes that can bind enzymes such as horseradish peroxidase and approaches such as tyramide signal amplification, fluorescence imaging using nucleic acid probes directly labeled with fluorophores, or hybridization of secondary nucleic acid probes to these primary probes, with the secondary probes detected via any of the above methods.

In some cases, the spatial positions may be determined at super resolutions, or at resolutions better than the wavelength of light or the diffraction limit (although in other embodiments, super resolutions are not required). Non-limiting examples include STORM (stochastic optical reconstruction microscopy), STED (stimulated emission depletion microscopy), NSOM (Near-field Scanning Optical Microscopy), 4Pi microscopy, SIM (Structured Illumination Microscopy), SMI (Spatially Modulated Illumination) microscopy, RESOLFT (Reversible Saturable Optically Linear Fluorescence Transition Microscopy), GSD (Ground State Depletion Microscopy), SSIM (Saturated Structured-Illumination Microscopy), SPDM (Spectral Precision Distance Microscopy), Photo-Activated Localization Microscopy (PALM), Fluorescence Photoactivation Localization Microscopy (FPALM), LIMON (3D Light Microscopical Nanosizing Microscopy), Super-resolution optical fluctuation imaging (SOFI), or the like. See, e.g., U.S. Pat. No. 7,838,302, issued Nov. 23, 2010, entitled “Sub-Diffraction Limit Image Resolution and Other Imaging Techniques,” by Zhuang, et al.; U.S. Pat. No. 8,564,792, issued Oct. 22, 2013, entitled “Sub-diffraction Limit Image Resolution in Three Dimensions,” by Zhuang, et al.; or WO 2013/090360, published Jun. 20, 2013, entitled “High Resolution Dual-Objective Microscopy,” by Zhuang, et al., each incorporated herein by reference in their entireties.

In one embodiment, the sample may be illuminated by single Gaussian mode laser lines. In some embodiments, the illumination profiled may be flattened by passing these laser lines through a multimode fiber that is vibrated via piezo-electric or other mechanical means. In some embodiments, the illumination profile may be flattened by passing single-mode, Gaussian beams through a variety of refractive beam shapers, such as the piShaper or a series of stacked Powell lenses. In yet another set of embodiments, the Gaussian beams may be passed through a variety of different diffusing elements, such as ground glass or engineered diffusers, which may be spun in some cases at high speeds to remove residual laser speckle. In yet another embodiment, laser illumination may be passed through a series of lenslet arrays to produce overlapping images of the illumination that approximate a flat illumination field.

In some embodiments, the centroids of the spatial positions of the entities may be determined. For example, a centroid of a signaling entity may be determined within an image or series of images using image analysis algorithms known to those of ordinary skill in the art. In some cases, the algorithms may be selected to determine non-overlapping single emitters and/or partially overlapping single emitters in a sample. Non-limiting examples of suitable techniques include a maximum likelihood algorithm, a least squares algorithm, a Bayesian algorithm, a compressed sensing algorithm, or the like. Combinations of these techniques may also be used in some cases.

In addition, the signaling entity may be inactivated in some cases. For example, in some embodiments, a first secondary nucleic acid probe containing a signaling entity may be applied to a sample that can recognize a first read sequence, then the first secondary nucleic acid probe can be inactivated before a second secondary nucleic acid probe is applied to the sample. If multiple signaling entities are used, the same or different techniques may be used to inactivate the signaling entities, and some or all of the multiple signaling entities may be inactivated, e.g., sequentially or simultaneously.

Inactivation may be caused by removal of the signaling entity (e.g., from the sample, or from the nucleic acid probe, etc.), and/or by chemically altering the signaling entity in some fashion, e.g., by photobleaching the signaling entity, bleaching or chemically altering the structure of the signaling entity, e.g., by reduction, etc.). For instance, in one set of embodiments, a fluorescent signaling entity may be inactivated by chemical or optical techniques such as oxidation, photobleaching, chemically bleaching, stringent washing or enzymatic digestion or reaction by exposure to an enzyme, dissociating the signaling entity from other components (e.g., a probe), chemical reaction of the signaling entity (e.g., to a reactant able to alter the structure of the signaling entity) or the like. For instance, bleaching may occur by exposure to oxygen, reducing agents, or the signaling entity could be chemically cleaved from the nucleic acid probe and washed away via fluid flow.

In some embodiments, various nucleic acid probes (including primary and/or secondary nucleic acid probes) may include one or more signaling entities. If more than one nucleic acid probe is used, the signaling entities may each by the same or different. In certain embodiments, a signaling entity is any entity able to emit light. For instance, in one embodiment, the signaling entity is fluorescent. In other embodiments, the signaling entity may be phosphorescent, radioactive, absorptive, etc. In some cases, the signaling entity is any entity that can be determined within a sample at relatively high resolutions, e.g., at resolutions better than the wavelength of visible light or the diffraction limit. The signaling entity may be, for example, a dye, a small molecule, a peptide or protein, or the like. The signaling entity may be a single molecule in some cases. If multiple secondary nucleic acid probes are used, the nucleic acid probes may comprise the same or different signaling entities.

Non-limiting examples of signaling entities include fluorescent entities (fluorophores) or phosphorescent entities, for example, cyanine dyes (e.g., Cy2, Cy3, Cy3B, Cy5, Cy5.5, Cy7, etc.), Alexa Fluor dyes, Atto dyes, photoswtichable dyes, photoactivatable dyes, fluorescent dyes, metal nanoparticles, semiconductor nanoparticles or “quantum dots”, fluorescent proteins such as GFP (Green Fluorescent Protein), or photoactivabale fluorescent proteins, such as PAGFP, PSCFP, PSCFP2, Dendra, Dendra2, EosFP, tdEos, mEos2, mEos3, PamCherry, PAtagRFP, mMaple, mMaple2, and mMaple3. Other suitable signaling entities are known to those of ordinary skill in the art. See, e.g., U.S. Pat. No. 7,838,302 or WO2015160690A1, each incorporated herein by reference in its entirety. In some cases, spectrally distinct fluorescent dyes may be used.

In one set of embodiments, the signaling entity may be attached to an oligonucleotide sequence via a bond that can be cleaved to release the signaling entity. In one set of embodiments, a fluorophore may be conjugated to an oligonucleotide via a cleavable bond, such as a photocleavable bond. Non-limiting examples of photocleavable bonds include, but are not limited to, 1-(2-nitrophenyl)ethyl, 2-nitrobenzyl, biotin phosphoramidite, acrylic phosphoramidite, diethylaminocoumarin, 1-(4,5-dimethoxy-2-nitrophenyl)ethyl, cyclo-dodecyl (dimethoxy-2-nitrophenyl)ethyl, 4-aminomethyl-3-nitrobenzyl, (4-nitro-3-(1-chlorocarbonyloxyethyl)phenyl)methyl-S-acetylthioic acid ester, (4-nitro-3-(1-thlorocarbonyloxyethyl)phenyl)methyl-3-(2-pyridyldithiopropionic acid) ester, 3-(4,4′-dimethoxytrityl)-1-(2-nitrophenyl)-propane-1,3-diol-[2-cyanoethyl-(N,N-diisopropyl)]-phosphoramidite, 1-[2-nitro-5-(6-trifluoroacetylcaproamidomethyl)phenyl]-ethyl-[2-cyano-ethyl-(N,N-diisopropyl)]-phosphoramidite, 1-[2-nitro-5-(6-(4,4′-dimethoxytrityloxy)butyramidomethyl)phenyl]-ethyl-[2-cyanoethyl-(N,N-diisopropyl)]-phosphoramidite, 1-[2-nitro-5-(6-(N-(4,4′-dimethoxytrityl))-biotinamidocaproamido-methyl)phenyl]-ethyl-[2-cyanoethyl-(N,N-diisopropyl)]-phosphoramidite, or similar linkers. In another set of embodiments, the fluorophore may be conjugated to an oligonucleotide via a disulfide bond. The disulfide bond may be cleaved by a variety of reducing agents such as, but not limited to, dithiothreitol, dithioerythritol, beta-mercaptoethanol, sodium borohydride, thioredoxin, glutaredoxin, trypsinogen, hydrazine, diisobutylaluminum hydride, oxalic acid, formic acid, ascorbic acid, phosphorous acid, tin chloride, glutathione, thioglycolate, 2,3-dimercaptopropanol, 2-mercaptoethylamine, 2-aminoethanol, tris(2-carboxyethyl)phosphine, bis(2-mercaptoethyl) sulfone, N,N′-dimethyl-N,N′-bis(mercaptoacetyl)hydrazine, 3-mercaptoproptionate, dimethylformamide, thiopropyl-agarose, tri-n-butylphosphine, cysteine, iron sulfate, sodium sulfite, phosphite, hypophosphite, phosphorothioate, or the like, and/or combinations of any of these. In another embodiment, the fluorophore may be conjugated to an oligonucleotide via one or more phosphorothioate modified nucleotides in which the sulfur modification replaces the bridging and/or non-bridging oxygen. The fluorophore may be cleaved from the oligonucleotide, in certain embodiments, via addition of compounds such as but not limited to iodoethanol, iodine mixed in ethanol, silver nitrate, or mercury chloride. In yet another set of embodiments, the signaling entity may be chemically inactivated through reduction or oxidation. For example, in one embodiment, a chromophore such as Cy5 or Cy7 may be reduced using sodium borohydride to a stable, non-fluorescence state. In still another set of embodiments, a fluorophore may be conjugated to an oligonucleotide via an azo bond, and the azo bond may be cleaved with 2-[(2-N-arylamino)phenylazo]pyridine. In yet another set of embodiments, a fluorophore may be conjugated to an oligonucleotide via a suitable nucleic acid segment that can be cleaved upon suitable exposure to DNAse, e.g., an exodeoxyribonuclease or an endodeoxyribonuclease. Examples include, but are not limited to, deoxyribonuclease I or deoxyribonuclease II. In one set of embodiments, the cleavage may occur via a restriction endonuclease. Non-limiting examples of potentially suitable restriction endonucleases include BamHI, BsrI, NotI, XmaI, PspAI, DpnI, MboI, MnlI, Eco57I, Ksp632I, DraIII, Ahall, SmaI, MluI, HpaI, Apal, BclI, BstEII, TaqI, EcoRI, SacI, HindII, HaeII, DraII, Tsp509I, Sau3AI, Pacd, etc. Over 3000 restriction enzymes have been studied in detail, and more than 600 of these are available commercially. In yet another set of embodiments, a fluorophore may be conjugated to biotin, and the oligonucleotide conjugated to avidin or streptavidin. An interaction between biotin and avidin or streptavidin allows the fluorophore to be conjugated to the oligonucleotide, while sufficient exposure to an excess of addition, free biotin could “outcompete” the linkage and thereby cause cleavage to occur. In addition, in another set of embodiments, the probes may be removed using corresponding “toe-hold-probes,” which comprise the same sequence as the probe, as well as an extra number of bases of homology to the encoding probes (e.g., 1-20 extra bases, for example, 5 extra bases). These probes may remove the labeled readout probe through a strand-displacement interaction.

As used herein, the term “light” generally refers to electromagnetic radiation, having any suitable wavelength (or equivalently, frequency). For instance, in some embodiments, the light may include wavelengths in the optical or visual range (for example, having a wavelength of between about 400 nm and about 700 nm, i.e., “visible light”), infrared wavelengths (for example, having a wavelength of between about 300 micrometers and 700 nm), ultraviolet wavelengths (for example, having a wavelength of between about 400 nm and about 10 nm), or the like. In certain cases, as discussed in detail below, more than one entity may be used, i.e., entities that are chemically different or distinct, for example, structurally. However, in other cases, the entities may be chemically identical or at least substantially chemically identical.

Another aspect of the invention is directed to a computer-implemented method. For instance, a computer and/or an automated system may be provided that is able to automatically and/or repetitively perform any of the methods described herein. As used herein, “automated” devices refer to devices that are able to operate without human direction, i.e., an automated device can perform a function during a period of time after any human has finished taking any action to promote the function, e.g., by entering instructions into a computer to start the process. Typically, automated equipment can perform repetitive functions after this point in time. The processing steps may also be recorded onto a machine-readable medium in some cases.

For example, in some cases, a computer may be used to control imaging of the sample, e.g., using fluorescence microscopy, STORM or other super-resolution techniques such as those described herein. In some cases, the computer may also control operations such as drift correction, physical registration, hybridization and cluster alignment in image analysis, cluster decoding (e.g., fluorescent cluster decoding), error detection or correction (e.g., as discussed herein), noise reduction, identification of foreground features from background features (such as noise or debris in images), or the like. As an example, the computer may be used to control activation and/or excitation of signaling entities within the sample, and/or the acquisition of images of the signaling entities. In one set of embodiments, a sample may be excited using light having various wavelengths and/or intensities, and the sequence of the wavelengths of light used to excite the sample may be correlated, using a computer, to the images acquired of the sample containing the signaling entities. For instance, the computer may apply light having various wavelengths and/or intensities to a sample to yield different average numbers of signaling entities in each region of interest (e.g., one activated entity per location, two activated entities per location, etc.). In some cases, this information may be used to construct an image and/or determine the locations of the signaling entities, in some cases at high resolutions, as noted above.

### VI. KITS

The present disclosure provides kits for performing the methods described herein. Kits are provided for preparing a FFPE tissue section samples and determining nucleic acid targets in the sample. In an embodiment, the kit includes anchor probes and anchor agents disclosed herein together with one or more other components. In an embodiment, the kit also includes labeling agents disclosed herein for identification of cell types or tissue morphology. In some embodiments, the kit includes a number of different labeling agents indicating of a tissue obtained from a particular disease (e.g., solid tumor cancer). In some embodiments, the kit also includes a set of nucleic acid probes (e.g., MERFISH probes described herein) together with one or more other components for MERFISH imaging.

The one or more other kit components can include one or more buffers; a nuclear counterstain; a whole RNA content counterstain; an imaging buffer; software; and other components. A kit can also include instructions for employing the kit components as well as the use of any other reagent not included in the kit. Instructions can include variations that can be implemented.

In certain embodiments provided herein are kit components and protocols for preparing FFPE tissue section samples for transcriptome analysis. In one embodiment, a kit comprises one or more of the following components: deparaffinization buffer, decrosslinking buffer, conditioning buffer, sample prep wash buffer, formamide wash buffer, gel embedding premix, clearing premix, gel coverslip, pre-anchoring activator, anchoring buffer and digestion premix. Also provided may be anchor probes for immobilizing target nucleic acid (e.g. RNA transcripts) and target probes and reagents thereof used to specifically detect the target nucleic acid in the prepared sample. In embodiments, kits are provided comprising at least a first anchoring agent and a second anchoring agent or anchor probe.

Exemplary kit components are described in Example 1.

### SPECIFIC EMBODIMENTS

The following embodiments according to the methods described herein are provided.

Embodiment 1 is a method of detecting nucleic acid targets in a tissue sample, comprising:
a. contacting a tissue sample containing nucleic acid targets with anchor probes specifically binding the nucleic acid targets;
b. immobilizing the nucleic acid target-bound anchor probes in at least part of the tissue sample within a gel;
c. clearing the tissue sample within the polymer gel by removing or degrading non-targets;
d. contacting the tissue sample with a plurality of nucleic acid probes capable of selectively binding the nucleic acid targets;
e. detecting the nucleic acid probes bound to the nucleic acid targets within the tissue sample.

Embodiment 2 is a method of detecting nucleic acid targets in a tissue sample, comprising:
a. contacting a tissue sample containing nucleic acid targets with an anchor agent comprises a first chemical moiety that can react with and/or modify an internal base of the nucleic acid targets and a second chemical moiety that can be incorporated into the polymer gel;
b. immobilizing the nucleic acid target-bound anchor agents in at least part of the tissue sample within a gel;
c. clearing the tissue sample within the polymer gel by removing or degrading non-targets;
d. contacting the tissue sample with a plurality of nucleic acid probes capable of selectively binding the nucleic acid targets;
e. detecting the nucleic acid probes bound to the nucleic acid targets within the tissue sample.

Embodiment 3 is a method of detecting nucleic acid targets in a tissue sample, comprising:
a. contacting a tissue sample containing nucleic acid targets with an anchor agent comprises a first chemical moiety that can react with and/or modify an internal base of the nucleic acid targets and a second chemical moiety that can be incorporated into the polymer gel;
b. contacting the tissue sample with anchor probes specifically binding the nucleic acid targets;
c. immobilizing the nucleic acid target-bound anchor probes or the nucleic acid target-bound anchor agents in at least part of the tissue sample within a gel;
d. clearing the tissue sample within the polymer gel by removing or degrading non-targets;
e. contacting the tissue sample with a plurality of nucleic acid probes capable of selectively binding the nucleic acid targets;
f. detecting the nucleic acid probes bound to the nucleic acid targets within the tissue sample.

Embodiment 4 is the method of any one of embodiments 1-3, further comprising producing codewords or barcodes based on a distribution of the bound nucleic acid probes within the sample.

Embodiment 5 is the method of embodiment 4, further comprising, for at least some of the codewords, matching the codewords to valid codewords in a codebook, optionally wherein, if no match is found, applying error correction to the codeword to form a valid codeword or discard the codeword.

Embodiment 6 is the method of any one of embodiments 1, 2, 4, and 5, wherein the step (c) of clearing is performed after steps (a) and (b).

Embodiment 7 is the method of any one of embodiments 3-6, wherein the step (d) of clearing is performed after steps (a)-(c).

Embodiment 8 is the method of any one of embodiments 1-7, wherein the steps are performed in the order recited.

Embodiment 9 is the method of any one of embodiments 1-8, wherein the tissue sample is a formalin-fixed paraffin-embedded (FFPE) tissue section.

Embodiment 10 is the method of any one of embodiments 1-8, wherein the tissue sample has been deparaffinized and rehydrated prior to step (a).

Embodiment 11 is the method of any one of embodiments 1-10, further comprising, prior to step (a), deparaffinization and rehydration of the tissue sample.

Embodiment 12 is the method of any one of embodiments 1-8, wherein the tissue sample is a fresh frozen tissue sample.

Embodiment 13 is the method of any one of embodiments 1-8, wherein the tissue sample is a fixed frozen tissue sample.

Embodiment 14 is the method of any one of embodiments 1-13, wherein the nucleic acid target is RNA.

Embodiment 15 is the method of any one of embodiments 1-13, wherein the nucleic acid target is DNA.

Embodiment 16 is the method of any one of embodiments 1-15, wherein the gel comprises polyacrylamide.

Embodiment 17 is the method of any one of embodiments 1-16, wherein at least some of the anchor probes comprises a poly-dT portion.

Embodiment 18 is the method of embodiment 17, wherein at least some of the anchor probes comprises alternating dT and locked dT portions.

Embodiment 19 is the method of any one of embodiments 16-18, wherein at least some of the anchor probes comprises an acrydite portion able to polymerize with the gel.

Embodiment 20 is the method of embodiment 19, wherein the acrydite portion is bound to the 5′ end of the anchor probe.

Embodiment 21 is the method of embodiment 19, wherein the acrydite portion is bound to the 3′ end of the anchor probe.

Embodiment 22 is the method of embodiment 19, wherein the acrydite portion is bound to an internal base of the anchor probe.

Embodiment 23 is the method of any one of embodiments 1 and 3-22, further comprising, prior to step (a), contacting the sample with an anchor agent comprises a first chemical moiety that can react with and/or modify an internal base of the nucleic acid targets and a second chemical moiety that can be incorporated into the polymer gel.

Embodiment 24 is the method of embodiment 23, wherein the step (b) of immobilizing further comprises polymerizing a gel within the tissue sample, wherein the second chemical moiety of the anchor agent is co-polymerized with the polymer gel.

Embodiment 25 is the method of embodiment 23 or 24, wherein the second chemical moiety of at least some of the anchor agents comprises an acrydite portion able to polymerize with the gel.

Embodiment 26 is the method of any one of embodiments 23-25, wherein at least some of the nucleic acid targets are immobilized within the gel via both the anchor probe and the anchoring agent bound to the nucleic acid target.

Embodiment 27 is the method of any one of embodiments 1-26, wherein the non-targets comprise proteins, lipids, DNA, RNA, or extracellular matrix.

Embodiment 28 is the method of any one of embodiments 1-27, wherein clearing the tissue sample comprises removing or degrading proteins from the sample.

Embodiment 29 is the method of any one of embodiments 1-28, wherein clearing the tissue sample comprises removing or degrading lipids from the sample.

Embodiment 30 is the method of any one of embodiments 1-29, wherein clearing the tissue sample comprises removing or degrading non-target DNA from the sample.

Embodiment 31 is the method of any one of embodiments 1-30, wherein clearing the tissue sample comprises removing or degrading extracellular matrix from the sample.

Embodiment 32 is the method of any one of embodiments 1-31, wherein clearing the tissue sample comprises exposing the sample to an enzyme able to degrade a protein.

Embodiment 33 is the method of any one of embodiments 1-32, wherein clearing the tissue sample comprises exposing the sample to an enzyme able to degrade DNA.

Embodiment 34 is the method of any one of embodiments 1-33, wherein clearing the tissue sample comprises exposing the sample to an enzyme able to degrade RNA.

Embodiment 35 is the method of any one of embodiments 1-34, wherein clearing the tissue sample comprises exposing the sample to an enzyme able to degrade sugars or sugar-modified biomolecules.

Embodiment 36 is the method of any one of embodiments 1-35, wherein clearing the tissue sample comprises exposing the sample to a detergent.

Embodiment 37 is the method of any one of embodiments 1-36, wherein clearing the tissue sample comprises exposing the gel to a proteinase.

Embodiment 38 is the method of embodiment 37, wherein the proteinase comprises proteinase K.

Embodiment 39 is the method of any one of embodiments 1-38, wherein clearing the tissue sample comprises exposing the gel to guanidine HCl.

Embodiment 40 is the method of any one of embodiments 1-39, wherein clearing the tissue sample comprises exposing the gel to Triton X-100 (polyethylene glycol p-(1,1,3,3-tetramethylbutyl)-phenyl ether).

Embodiment 41 is the method of any one of embodiments 1-40, wherein clearing the tissue sample comprises exposing the gel to sodium dodecyl sulfate.

Embodiment 42 is the method of any one of embodiments 1-41, wherein clearing the tissue sample comprises exposing the gel to ethylenediaminetetraacetic acid.

Embodiment 43 is the method of any one of embodiments 1-42, wherein the plurality of nucleic acid probes comprises smFISH probes.

Embodiment 44 is the method of any one of embodiments 1-43, wherein the plurality of nucleic acid probes comprises MERFISH probes.

Embodiment 45 is the method of any one of embodiments 1-44, wherein the step (d) further comprises amplification of the nucleic acid probes.

Embodiment 46 is the method of any one of embodiments 1-45, wherein the detecting comprises imaging using optical microscopy.

Embodiment 47 is the method of any one of embodiments 1-46, wherein the detecting comprises imaging using fluorescence microscopy.

Embodiment 48 is the method of embodiment 47, comprising imaging using epi-fluorescence microscopy, total-internal-reflectance microscopy, highly inclined thin-illumination (HILO) microscopy, light-sheet microscopy, scanning confocal microscopy, scanning line confocal microscopy, spinning disk confocal microscopy, or other comparable conventional microscopy techniques.

Embodiment 49 is the method of embodiment 47, comprising imaging using multiplexed fluorescence in situ hybridization.

Embodiment 50 is the method of embodiment 47, comprising imaging using multiplexed error robust fluorescence in situ hybridization (MERFISH).

Embodiment 51 is the method of any one of embodiments 47-50, comprising imaging using multiple rounds of fluorescence in situ hybridization.

Embodiment 52 is the method of any one of embodiments 1-51, wherein the nucleic acid probes comprise a targeting sequence and one or more read sequences.

Embodiment 53 is the method of embodiment 52, further comprising determining read sequences based on determining binding of the read sequences bound to target nucleic acid targets.

Embodiment 54 is the method of embodiment 53, wherein the codewords or barcodes are created based on determination of the read sequences within the gel.

Embodiment 55 is the method of any one of embodiments 52-54, wherein the read sequences are taken from a set of orthogonal sequences, which have a homology of less than 15 base pairs with one another and with the nucleic acid species in a sample.

Embodiment 56 is the method of any one of embodiments 4-55, wherein each of the codeword represents one of the plurality of different nucleic acid targets and comprises multiple binary values 1 and 0, wherein a value of 1 is obtained when the signal is detected at a respective location within the sample while a value of 0 is obtained when the signal is not detected.

Embodiment 57 is the method of any one of embodiments 4-56, wherein the codewords representing the plurality of different nucleic acid targets at locations within the sample are produced, wherein each of the codeword represents one of the plurality of different nucleic acid targets and comprises multiple binary values 1 and 0, wherein a value of 1 is obtained when the signal is detected from one of the plurality of readout probe-hybridized complexes or one of the different plurality of readout probe-hybridized complexes at a respective location within the sample while a value of 0 is obtained when the signal is not detected from one of the plurality of readout probe-hybridized complexes or one of the different plurality of readout probe-hybridized complexes at the respective location within the sample.

Embodiment 58 is the method of any one of embodiments 5-57, wherein the codebook comprises the valid codewords of the plurality of nucleic acid targets.

Embodiment 59 is the method of any one of embodiments 5-58, wherein the step (h) of matching the codewords with valid codewords in a cod book comprises comparing the codeword to valid codewords in a codebook, and if the codeword is not matched with one of the valid codewords in the codebook, applying an error detection or correction system, matching the codeword with another of the valid codewords in the codebook, or discarding the codeword, wherein the codebook comprises the valid codewords of the plurality of nucleic acid targets.

Embodiment 60 is the method of any one of embodiments 1-59, wherein the tissue sample has been contacted with at least one labeling agent for labeling at least one cellular component prior to step (a).

Embodiment 61 is the method of any one of embodiments 1-60, further comprising, prior to step (a), contacting the tissue sample with at least one labeling reagent for labeling at least one cellular component prior to step (a).

Embodiment 62 is the method of embodiment 60 or 61, wherein the at least one labeling reagent comprises at least three oligonucleotide-conjugated labeling probes, each comprising an oligonucleotide conjugated to an anchor moiety that can be attached to a gel and (2) a binding moiety that can bind to a cellular component, wherein the binding moiety comprises (i) a protein-binding moiety that can bind to a cellular protein component; (ii) a carbohydrate-binding moiety that can bind to a cellular carbohydrate component; or (iii) a chemical binding moiety that can bind to or incorporate into a cellular component.

In some preferred embodiments, this disclosure provides methods of anchoring target nucleic acid within a matrix and clearing non-target cellular components comprising: contacting a formalin fixed paraffin embedded (FFPE) tissue sample with at least two anchoring agents, wherein the first anchoring agent forms a covalent bond with the target nucleic acid and the second anchoring agent comprises an oligonucleotide that hybridizes with the target nucleic acid; embedding the sample in a polymer matrix wherein the first and second anchoring agents each form a covalent bond with the polymer matrix; and, clearing the non-target cellular components from the polymer matrix wherein the target nucleic acid remains anchored in the polymer matrix to form a matrix anchored target nucleic acid sample. In some preferred embodiments, the first anchoring agent is an alkylating agent. In some preferred embodiments, the alkylating agent is selected from the group consisting of Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa and Trabectedin. In some preferred embodiments, the second anchoring agent comprises alternating dT and locked dT portions that hybridizes with the target nucleic acid. In some preferred embodiments, the second anchoring agent comprises a poly-dT portion that hybridizes to the target nucleic acid. In some preferred embodiments, the first anchoring agent and the second anchoring agent each comprise an acrydite moiety that covalently binds the polymer matrix. In some preferred embodiments, the first anchoring agent is an alkylating agent derivatized with an acrydite moiety. In some preferred embodiments, the second anchoring agent comprises a poly-dT portion that hybridizes to the target nucleic acid and an acrydite moiety that covalently binds the polymer matrix. In some preferred embodiments, the target nucleic acid is RNA. In some preferred embodiments, the target nucleic acid is DNA. In some preferred embodiments, the method further comprises contacting the anchored target nucleic acid sample with one or more primary oligonucleotide probes that hybridize to the target nucleic acids. In some preferred embodiments, the primary oligonucleotide probes are single molecule (sm)FISH probes or multiplexed error robust fluorescence in situ hybridization (MERFISH) probes. In some preferred embodiments, the one or more primary oligonucleotide probes comprise a first portion comprising a target sequence and a second portion comprising one or more read sequences. In some preferred embodiments, the method further comprises determining read sequences based on contacting the one or more primary oligonucleotide probes with a plurality of secondary nucleic acid probes comprising a recognition sequence that hybridizes to the read sequence of the primary nucleic acid probe. In some preferred embodiments, the method further comprises imaging using multiplexed fluorescence in situ hybridization comprising contacting the anchored target nucleic acid sample with one or more primary oligonucleotide probes that hybridize to the target nucleic acids and comprising one or more sequential steps of adding a plurality of secondary nucleic acid probes comprising a label moiety. In some preferred embodiments, the method further comprises imaging using multiplexed error robust fluorescence in situ hybridization (MERFISH) probes comprising contacting the anchored target nucleic acid sample with one or more MERFISH probes that hybridize to the target nucleic acids. In some preferred embodiments, the method comprises imaging using multiple rounds of fluorescence in situ hybridization wherein, in each round, one or more different secondary nucleic acid probes, each conjugated to a spectrally distinct fluorescent label are used to readout out multiple readout sequences simultaneously.

In some preferred embodiments, this disclosure provides methods of anchoring target RNA within a matrix and clearing non-target cellular components comprising: contacting a formalin fixed paraffin embedded (FFPE) tissue sample with at least two anchoring agents, wherein the first anchoring agent comprises an alkylating agent that forms a covalent bond with the target nucleic acid and the second anchoring agent comprises a polyT sequence that is complementary and hybridizes to the target RNA and wherein the first anchoring agent and the second anchoring agent each comprise an acrydite moiety that covalently binds to the matrix; embedding the sample in a polymer matrix wherein the first and second anchoring agents each form a covalent bond with the polymer matrix; and, clearing the non-target cellular components from the polymer matrix wherein the target RNA remains anchored in the polymer matrix to form a matrix anchored target RNA sample. In some preferred embodiments, the alkylating agent is selected from the group consisting of Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa and Trabectedin. In some preferred embodiments, the second anchoring agent comprises alternating dT and locked dT portions that hybridizes with the target RNA. In some preferred embodiments, the methods further comprise contacting the anchored target nucleic acid sample with one or more primary oligonucleotide probes that hybridize to the target nucleic acids. In some preferred embodiments, the primary oligonucleotide probes are single molecule (sm)FISH probes or multiplexed error robust fluorescence in situ hybridization (MERFISH) probes. In some preferred embodiments, the one or more primary oligonucleotide probes comprise a first portion comprising a target sequence and a second portion comprising one or more read sequences. In some preferred embodiments, the methods further comprise determining read sequences based on contacting the one or more primary oligonucleotide probes with a plurality of secondary nucleic acid probes comprising a recognition sequence that hybridizes to the read sequence of the primary nucleic acid probe. In some preferred embodiments, the methods further comprise imaging using multiplexed fluorescence in situ hybridization comprising contacting the anchored target nucleic acid sample with one or more primary oligonucleotide probes that hybridize to the target nucleic acids and comprising one or more sequential steps of adding a plurality of secondary nucleic acid probes comprising a label moiety. In some preferred embodiments, the methods further comprise imaging using multiplexed error robust fluorescence in situ hybridization (MERFISH) probes comprising contacting the anchored target nucleic acid sample with one or more MERFISH probes that hybridize to the target nucleic acids. In some preferred embodiments, the methods further imaging using multiple rounds of fluorescence in situ hybridization wherein, in each round, one or more different secondary nucleic acid probes, each conjugated to a spectrally distinct fluorescent label are used to readout out multiple readout sequences simultaneously.

In some preferred embodiments, this disclosure provides methods for imaging target nucleic acid within a matrix and clearing non-target cellular components comprising: contacting a formalin fixed paraffin embedded (FFPE) tissue sample with at least two anchoring agents, wherein the first anchoring agent forms a covalent bond with the target nucleic acid and the second anchoring agent comprises an oligonucleotide that hybridizes with the target nucleic acid; embedding the sample in a polymer matrix wherein the first and second anchoring agents each form a covalent bond with the polymer matrix; clearing the non-target cellular components from the polymer matrix wherein the target nucleic acid remains anchored in the polymer matrix to form a matrix anchored target nucleic acid sample; and, contacting the anchored target nucleic acid sample with one or more primary oligonucleotide probes that hybridize to the target nucleic acids and a plurality of secondary nucleic acid probes comprising a fluorescent label and a recognition sequence that hybridizes to a sequence of the primary nucleic acid probe and imaging the target nucleic acids. In some preferred embodiments, the first anchoring agent is an alkylating agent. In some preferred embodiments, the alkylating agent is selected from the group consisting of Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa and Trabectedin. In some preferred embodiments, the second anchoring agent comprises alternating dT and locked dT portions that hybridizes with the target nucleic acid. In some preferred embodiments, the second anchoring agent comprises a poly-dT portion that hybridizes to the target nucleic acid. In some preferred embodiments, the first anchoring agent and the second anchoring agent each comprise an acrydite moiety that covalently binds the polymer matrix. In some preferred embodiments, the first anchoring agent is an alkylating agent derivatized with an acrydite moiety. In some preferred embodiments, the second anchoring agent comprises a poly-dT portion that hybridizes to the target nucleic acid and an acrydite moiety that covalently binds the polymer matrix. In some preferred embodiments, the target nucleic acid is RNA. In some preferred embodiments, the target nucleic acid is DNA. In some preferred embodiments, the primary oligonucleotide probes are single molecule (sm)FISH probes or multiplexed error robust fluorescence in situ hybridization (MERFISH) probes. In some preferred embodiments, the one or more primary oligonucleotide probes comprise a first portion comprising a target sequence and a second portion comprising one or more read sequences. In some preferred embodiments, the methods further comprise determining read sequences based on contacting the one or more primary oligonucleotide probes with a plurality of secondary nucleic acid probes comprising a recognition sequence that hybridizes to the read sequence of the primary nucleic acid probe. In some preferred embodiments, the secondary nucleic acid probes are added in one or more sequential steps and imaging performed between each sequential round of adding the secondary nucleic acid probes. In some preferred embodiments, the methods further comprise imaging using multiplexed error robust fluorescence in situ hybridization (MERFISH) probes comprising contacting the anchored target nucleic acid sample with one or more MERFISH probes that hybridize to the target nucleic acids. In some preferred embodiments, the methods further comprise imaging using multiple rounds of fluorescence in situ hybridization wherein, in each round, one or more different secondary nucleic acid probes, each conjugated to a spectrally distinct fluorescent label are used to readout out multiple readout sequences simultaneously.

In some preferred embodiments, this disclosure provides methods for imaging target RNA within a matrix and clearing non-target cellular components comprising: contacting a formalin fixed paraffin embedded (FFPE) tissue sample with at least two anchoring agents, wherein the first anchoring agent comprises an alkylating agent that forms a covalent bond with the target nucleic acid and the second anchoring agent comprises a polyT sequence that is complementary and hybridizes to the target RNA and wherein the first anchoring agent and the second anchoring agent each comprise an acrydite moiety that covalently binds to the matrix; embedding the sample in a polymer matrix wherein the first and second anchoring agents each form a covalent bond with the polymer matrix; clearing the non-target cellular components from the polymer matrix wherein the target RNA remains anchored in the polymer matrix to form a matrix anchored target RNA sample; and, contacting the anchored target RNA sample with one or more primary oligonucleotide probes that hybridize to the target RNA and a plurality of secondary nucleic acid probes comprising a fluorescent label and a recognition sequence that hybridizes to a sequence of the primary nucleic acid probe and imaging the target nucleic acids. In some preferred embodiments, the alkylating agent is selected from the group consisting of Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa and Trabectedin. In some preferred embodiments, the second anchoring agent comprises alternating dT and locked dT portions that hybridizes with the target nucleic acid. In some preferred embodiments, the primary oligonucleotide probes are single molecule (sm)FISH probes or multiplexed error robust fluorescence in situ hybridization (MERFISH) probes. In some preferred embodiments, the one or more primary oligonucleotide probes comprise a first portion comprising a target sequence and a second portion comprising one or more read sequences. In some preferred embodiments, the methods further comprise determining read sequences based on contacting the one or more primary oligonucleotide probes with a plurality of secondary nucleic acid probes comprising a recognition sequence that hybridizes to the read sequence of the primary nucleic acid probe. In some preferred embodiments, the secondary nucleic acid probes are added in one or more sequential steps and imaging performed between each sequential round of adding the secondary nucleic acid probes. In some preferred embodiments, the methods further comprise imaging using multiplexed error robust fluorescence in situ hybridization (MERFISH) probes comprising contacting the anchored target nucleic acid sample with one or more MERFISH probes that hybridize to the target nucleic acids. In some preferred embodiments, the methods further comprise imaging using multiple rounds of fluorescence in situ hybridization wherein, in each round, one or more different secondary nucleic acid probes, each conjugated to a spectrally distinct fluorescent label are used to readout out multiple readout sequences simultaneously.

Although embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the embodiments, specific terminology will be resorted to for the sake of clarity.

It must also be noted that, as used in the specification and the appended claims, the singular forms “a,” “an” and “the” include plural references unless the context clearly dictates otherwise. For example, reference to “a probe” is intended to also include a plurality of probes and reference to “a target” is intended to also include a plurality of targets and the like.

The term “or” is used herein in the inclusive sense, i.e., equivalent to “and/or” unless the context clearly requires otherwise.

Numeric ranges are inclusive of the numbers defining the range. Measured and measurable values are understood to be approximate, taking into account significant digits and the error associated with the measurement. Accordingly, unless indicated to the contrary, the numerical parameters set forth in the following description and appended claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The term “about” or “approximately” means an acceptable error for a particular value as determined by one of ordinary skill in the art, which depends in part on how the value is measured or determined, or a degree of variation that does not substantially affect the properties of the described subject matter.

The use of “comprise”, “comprises”, “comprising”, “contain”, “contains”, “containing”, “include”, “includes”, and “including” is not intended to be limiting, and means that at least the named compound, element, particle, or method step is present in the composition or article or method, but does not exclude the presence of other compounds, materials, particles, method steps, even if the other such compounds, material, particles, method steps have the same function as what is named.

Also, in describing the embodiments, terminology will be resorted to for the sake of clarity. It is intended that each term contemplates its broadest meaning as understood by those skilled in the art and includes all technical equivalents which operate in a similar manner to accomplish a similar purpose.

It is also to be understood that the mention of one or more method steps does not preclude the presence of additional method steps or intervening method steps between those steps expressly identified. Similarly, it is also to be understood that the mention of one or more components in a fabric or system does not preclude the presence of additional components or intervening components between those components expressly identified.

The skilled artisan will understand that the figures, described above, and example, described below, are for illustration purposes only. Neither the figures nor the examples are intended to limit the scope of the disclosed teachings in any way.

### VII. EXAMPLES

The following examples are provided to illustrate certain disclosed embodiments and are not to be construed as limiting the scope of this disclosure in any way.

### Example 1. In Situ Single-Cell Transcriptomic Imaging Through MERFISH in FFPE Samples (“Protocol A”)

This example illustrates a sample protocol (“Protocol A”) for preparing FFPE samples for MERFISH imaging. It is understood this Protocol A can be used with any downstream in situ imaging, not just MERFISH imaging, and for anchoring fragmented RNA and that this example is provided for illustrative purposes only and not to imply any limitation on the methods described herein. Protocol A and comparative protocol (“Protocol B”) (in which the MERFISH and one more anchoring agents are added to the sample prior to clearing FFPE and fresh frozen samples) are illustrated in FIG. 1.

In a certain embodiments, Protocol A can be carried out as described below:
1) Fiducial coating on a slide (e.g., MERSCOPE Slide, Vizgen, #20400001) that enables image registration for multiple rounds of imaging:
   a. Make a bead solution (e.g., Spherotech fluorescent beads, Catalog number: FP-0252-2) at 1:500 dilution in PBS.
   b. Add 500 μl the bead solution onto the center of a bead pre-coated coverslip to cover about 80% of the whole area of the coverslip. Avoid the bead solution flowing beneath the coverslip. Incubate at room temperature for 10 minutes.
   c. Aspirate the bead solution.
   d. 500 μl PBS wash once and aspirate the PBS. Avoid the bead solution flowing down the coverslip.
   e. Add another 500 μl PBS to the center of the coverslip.
2) Sectioning of FFPE slices onto fiducial coated coverslip
   a. Immediately before sectioning of FFPE slices, aspirate the PBS solution.
   b. Place 4-5 μm in thickness of FFPE slice(s) onto the prepared coverslip.
   c. Dry the coverslip at room temperature for 2-3 minutes.
   d. Dry the coverslip at 50-55° C. in an oven for 10 minutes.
   e. Completely remove all remaining water drop(s).
   f. The FFPE coverslips can be used right away or stored in −20 degree freezer for 1-2 months.
3) Deparaffinization and rehydration of FFPE slices
   a. Add 400 μl deparaffinization solution (e.g., Zymo Research, D3067-1-20) onto the center of the coverslip. Allow the solution to gradually cover the whole coverslip.
   b. Place the coverslip into an oven at 50-55 degree for 5 minutes.
   c. Aspirate the solution.
   d. Add another 350 ul deparaffinization solution onto the center of the coverslip. Allow the solution to cover the whole coverslip.
   e. Incubate at room temperature for 2 minutes.
   f. Aspirate the solution.
   g. Add 5 ml 100% ethanol to the dish with the coverslip to rinse, and aspirate.
   h. Add 5 ml 100% ethanol to the dish with the coverslip and incubate at room temperature for 2 minutes, and aspirate.
   i. Repeat step 3(h) one more time.
   j. Add 5 ml 90% ethanol to the dish with the coverslip and incubate at room temperature for 5 minutes, and aspirate.
   k. Add 5 ml 70% ethanol to the dish with the coverslip and incubate at room temperature for 5 minutes, and aspirate.
   l. The coverslip can be stored in 70% ethanol for up to one month.
4) Antigen retrieval of FFPE slices
   a. Add 5 ml citric base antigen retrieval buffer to rinse and aspirate.
   b. Add 5 ml citric base antigen retrieval buffer. Place the dish into an oven at 90-95 degree for 15 minutes.
   c. Place the dish on the bench at room temperature for 5 minutes and aspirate.
5) Immunostaining of protein biomarkers for cell segmentation and protein imaging. In this step cell morphology and protein stain is performed.
   a. Add 5 ml PBS into the dish to rinse, and aspirate.
   b. Add 100 μl blocking solution containing 3% bovine serum albumin (BSA) to cover the whole tissue. Incubate at room temperature for 45-60 minutes. Aspiration.
   c. Add 100 μl blocking solution containing 3% BSA+primary antibody for Cell boundary 3 or other antibodies at dilutions according to the pre-determined concentrations. The solution needs to cover the whole tissue. Incubate at room temperature for 60 minutes.
   d. Add 5 ml PBS into the dish. Incubate on the bench for 5 minutes. Shake occasionally, and aspirate.
   e. Repeat step 5(d) once.
   f. Add 100 μl blocking solution containing 3% BSA+secondary antibody for cell boundary/cell morphology staining according to the pre-determined concentrations. Incubate at room temperature for 60 minutes.
   g. Add 5 ml PBS into the dish. Incubate on the bench for 5 minutes. Shake occasionally, and aspirate.
   h. Repeat step 5(g) twice.
6) FFPE slices anchoring pretreatment to prime the tissue for anchoring. In this step a first anchoring agent is added to the sample and forms a covalent bond with the nucleic acid
   a. Add 4 ml anchoring pretreatment dilution buffer into the dish to rinse and aspirate.
   b. Add 4 ml anchoring pretreatment dilution buffer into the dish. Incubate at a 37 degree incubator for 30 minutes, and aspirate.
   c. Add 100 μl anchoring pretreatment solution on the tissue to cover the whole tissue. Incubate at a 37° C. incubator for 2 hours.
7) Anchoring of RNA molecules with polyT probe. In this step a second anchoring agent or anchor probe is added to the sample and hybridizes with the poly-A tail of the mRNA present in the sample.
   a. Add 5 ml sample prep wash buffer into the dish to rinse, and aspirate.
   b. Add 5 ml formamide wash buffer into the dish. Incubate at a 37 degree incubator for 30 minutes. Aspirate the solution.
   c. Add 75 μl PolyT anchoring buffer onto the center of the tissue. Use a piece of parafilm 2×2 cm to spread and cover the whole tissue. Incubate at a 37 degree incubator for overnight (at least >16 hours).
   d. Add 5 ml formamide wash buffer into the dish. Incubate at a 47 degree incubator for 15 minutes, and aspirate.
8) Gel embedding and tissue clearing to remove autofluorescence background. In this step the mRNA is immobilized in the gel when the first and second anchoring agents each form covalent bonds with the polyacrylamide gel and the non-immobilized cellular components are removed providing a gel immobilized target nucleic acid sample.
   a. Add 5 ml sample prep wash buffer into the dish to rinse. Aspiration.
   b. Clean a Gel Coverslip (Vizgen, Inc) by spraying with RnaseZap solution and wiping with a Kimwipe, followed by spraying 70% ethanol and wiping with a Kimwipe.
   c. Add 50 μL Gel Slick Solution (VWR, Catalog number 12001-812) onto the Gel Coverslip, wipe gently with a Kimwipe to spread the Gel Slick.
   d. Prepare gel embedding solution, including polyacrylamide, 0% w/v ammonium persulfate solution, and N,N,N′,N′-tetramethylethylenediamine.
   e. Aspirate the sample prep wash buffer. Retain 100 μL gel embedding solution in a small tube. Add the remainder of the 5 mL embedding solution, incubate at room temperature for 1 min.
   f. Using a pipette, transfer the majority of the embedding solution to a waste tube (to monitor the gel formation).
   g. Aspirate to dry the slide, leaving just enough liquid to cover the tissue section.
   h. Add 50 μL of the retained gel embedding solution on the tissue section.
   i. Place the tips of one pair of tweezers on an area of the MERSCOPE Slide without touching the tissue section. Use tweezers to pick up the 20-mm Gel Slick-treated Gel Coverslip. With the Gel Slick-treated side facing down toward the tissue, place the edge of the Gel Coverslip against the tweezer tips resting on the MERSCOPE Slide, creating stability, and slowly lower the Gel Coverslip onto the tissue section to spread the Gel Embedding Solution. If needed, adjust the Gel Coverslip so it is positioned in the center of the MERSCOPE Slide. Gently press the Gel Coverslip to squeeze out excess Gel Embedding Solution, and remove the extra gel embedding solution by aspiration.
   j. Incubate at room temperature for 1.5 h.
   k. Ensure eye protection is worn during this step. Gently lift the 20-mm Gel Slick-treated Gel Coverslip with the sharp tip of a Hobby Blade and discard the Gel Coverslip appropriately.
   l. Warm clearing premix (containing SDS, 2×SSC and Trition-x 100) at 37° C. for 30 min before use. The clearing premix should be a clear solution before use. If the solution is cloudy, warm until the solution becomes clear. Prepare clearing solution including the clearing premix and proteinase K (100:1 volume ratio).
   m. (Optional) For the resistant tissue, add 200 μl digestion premix containing mixtures of enzymes that can digest the connective tissue 200 μl+10 μl Rnase inhibitor onto the gel. Incubate in a 37 degree incubator for 1-6 hours.
   n. Add 5 ml clearing solution to the dish. Seal the dish with a piece of parafilm. Incubate in a 47 degree incubator for 18-24 hours. After 18 hours incubation, monitor the tissue transparency. If the tissue becomes clearing, go to the next step.
   o. Photobleach for 3-4 hours. (Note: this Photobleaching step can be performed after the “step 3)” above)
   p. The sample can be stored in 5 ml clearing solution in a 37 degree incubator for up to 1 week.
9) Hybridization with MERFISH encoding probes (“primary oligonucleotide probes”) for imaging of the anchored mRNA, and which are designed to hybridize to a plurality of target nucleic acid within the anchored mRNA.
   a. Add 5 ml sample prep wash buffer. Shake on a shaker at room temperature for 5 minutes, and aspirate.
   b. Repeat step 9(a) three times.
   c. Add 5 ml formamide wash buffer into the dish. Incubate at a 37 degree incubator for 30 minutes. Aspirate the solution.
   d. Add 80 μl MERFISH encoding probe (“primary oligonucleotide probes”) onto the gel with the anchored mRNA. Use a piece of parafilm 2×2 cm to spread and cover the whole tissue. Incubate at a 37 degree incubator for two days (36-48 hours).
10) Washing to remove excess or unbound primary oligonucleotide probes:
   a. Add 5 ml Formamide Wash Buffer into the dish. Incubate at a 47 degree incubator for 30 minutes, and aspirate.
   b. Repeat 10(a) once.
   c. The sample can be stored in 5 ml clearing solution in a 37 degree incubator for up to 1 week. After storing, the sample need to be washed with 5 ml sample prep wash buffer. Shake on a shaker at room temperature for 5 minutes and aspirate. Repeat wash with 5 ml sample prep wash buffer once.
11) (Optional) Signal enhancing (Amplification)
   a. Add 100 ul enhancer solution to the gel with the tissue. Incubate at a 37 degree incubator for overnight (>16 hours).
   b. Add 5 ml formamide wash buffer into the dish. Incubate on the bench at room temperature for 5 minutes.
   c. Add 5 ml formamide wash buffer into the dish. Incubate on the bench at room temperature for 10 minutes.
   d. Rinse with 5 ml sample prep wash buffer.
12) Data acquisition and analysis
   a. Add 3 mL DAPI and PolyT Staining Reagent, incubate 15 min on a rocker.
   b. Wash with 5 mL formamide wash buffer, incubate 10 min.
   c. Wash with 5 mL sample prep wash buffer.
   d. Place the sample coverslip on a MERSCOPE (Vizgen, Inc.) and the secondary nucleic acid probes (“read out probes”) are added sequentially to acquire data and then do an analysis.

### Example 2. In Situ Single-Cell Transcriptomic Imaging Through MERFISH in FFPE Samples

This example illustrates the results of MERFISH imaging in FFPE samples following the target immobilization and clearing method according to the present disclosure. Following immobilization of the target nucleic acid and clearing non-target cellular components primary nucleic probes are added, which are designed to hybridize to the immobilized target nucleic acid, and then addition of read out probes which bind to complementary sequences (“read sequences”) of the primary nucleic acid probes and which comprise a fluorescent label. Further details of the method are provided above in Example 1.

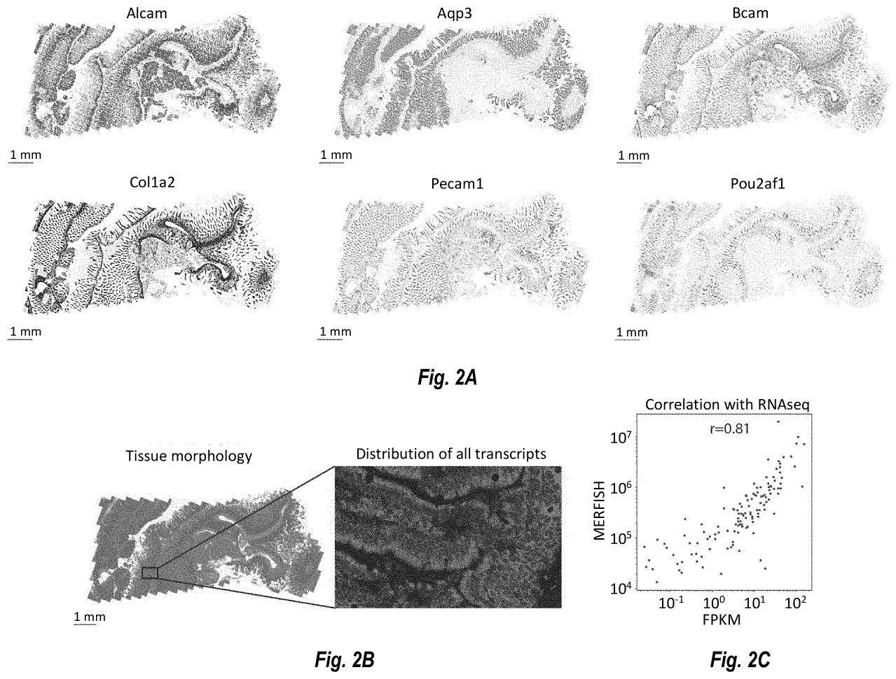

FIGS. 2A-C show MERFISH imaging with 128-plex gene panel in FFPE mouse small intestine. FIG. 2A shows spatial distributions of select genes across the tissue. FIG. 2B shows tissue morphology visualized by select transcripts (left) and distribution of all transcripts in zoomed in region.

To determine the accuracy of these measurements, the copy number per gene as determined via MERFISH (“MERFISH count” “MERFISH” as in FIG. 2C) for the tissue sections was compared with the abundance as determined via RNA-seq as measured in FPKM derived from the same tissue samples. As shown in FIG. 2C, the MERFISH count was strongly correlated with bulk RNA sequencing FPKM data (r=0.81), indicating the measurement is quantitative and highly accurate.

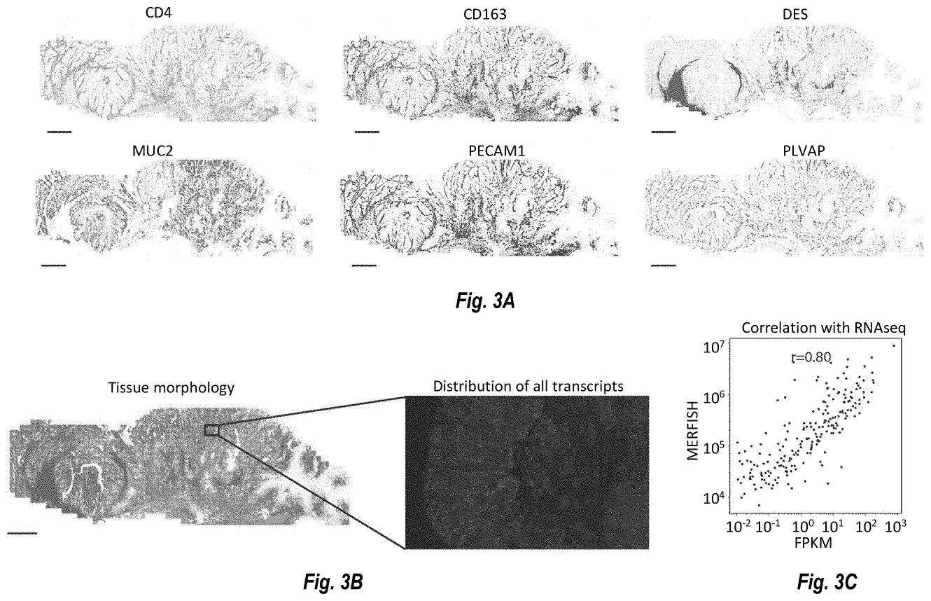

Similarly, FIG. 3 shows MERFISH imaging with 244-plex gene panel in FFPE human colon cancer. FIG. 3A shows spatial distributions of select genes across the tissue. FIG. 3B shows tissue morphology visualized by select transcripts (left) and distribution of all transcripts in zoomed in region (right). As shown in FIG. 3C, the MERFISH counts were strongly corrected with bulk RNA sequencing FPKM data (r=0.80), indicating the measurement is quantitative and highly accurate.

FIG. 4 also shows MERFISH imaging with 483-plex gene panel in FFPE mouse brain. FIG. 4A shows spatial distributions of select genes across the tissue. FIG. 4B shows tissue morphology visualized by select transcripts (left) and distribution of all transcripts in zoomed in region (right). As shown in FIG. 4C, the MERFISH counts were strongly corrected with bulk RNA sequencing FPKM data (r=0.88), indicating the measurement is quantitative and highly accurate.

### Example 3. Comparison to Other Imprinting and Tissue Clearing Protocol

This example provides comparison of the sample preparation according to certain embodiments of the present disclosure (“Protocol A”) to a comparative protocol (“Protocol B”) in which the MERFISH (encoding or primary oligonucleotide probes) and anchor probes are added to the sample prior to clearing FFPE and fresh frozen samples (FIG. 1). FFPE mouse small intestine samples were processed with Protocol A or Protocol B. Fresh frozen mouse small intestine samples also were processed with Protocol B. All samples were then imaged via a MERSCOPE protocol (i.e., addition of MERFISH encoding probes and readout probes)

FIG. 5 shows average counts of transcripts per field of view (FOV) for both conditions, indicating that the samples prepared according to Protocol A showed higher level of detection of transcripts indicating more of the nucleic acid in the sample was anchored in the matrix and available for hybridization to the encoding probes.

### Example 4. MERFISH Imaging in Human FFPE Tissue Samples

This example illustrates MERFISH measurements of human FFPE tissue samples prepared according to Protocol A the present disclosure.

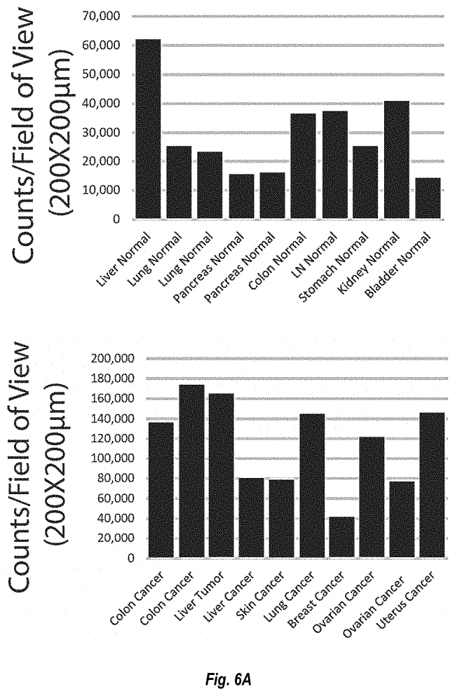

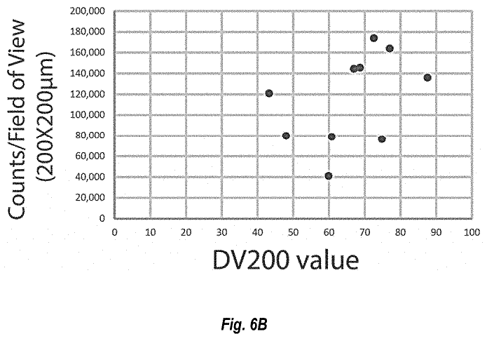

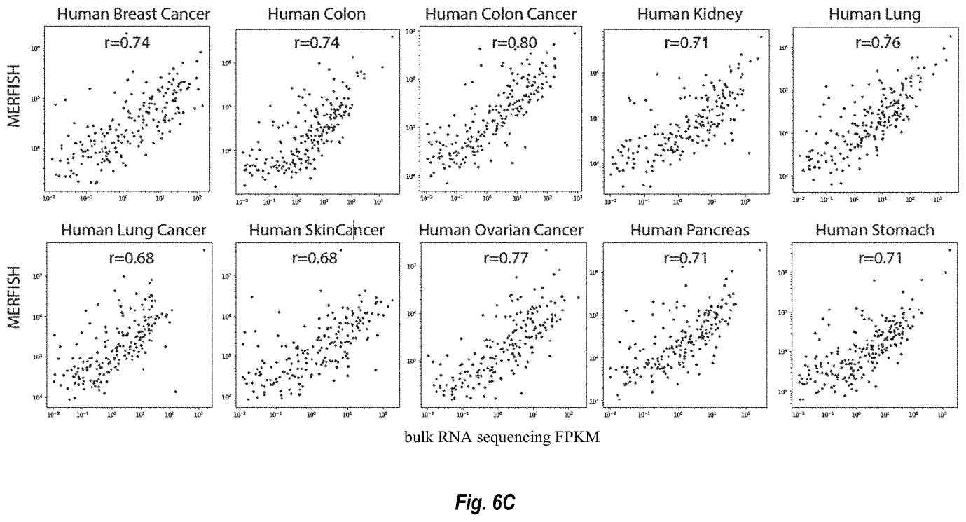

FIG. 6 shows MERFISH imaging in 15 different archival human FFPE samples with 244-plex gene panel. For each dataset, 1000-2000 fields of views were captured, generating 10s-100s million counts per tissue slice. FIG. 6A, top plot, shows average counts per field of view with an area size of 200×200 μm, indicating the workflow works robustly across a wide range of FFPE samples obtained from normal human tissue (top) and from human tumor (bottom). FIG. 6B shows that MERFISH data quality is correlated with sample's RNA quality, as indicated by DV200 value. DV200 is the percent of RNA fragments >200 nucleotides in samples. As shown in FIG. 6C, the MERFISH counts were strongly corrected with bulk RNA sequencing FPKM data (r=0.88) across various human tissue samples.

### Example 5. Comparison in Various Human FFPE Samples

This example provides comparison of MEFISH imaging of the human FFPE samples according to certain embodiments of the present disclosure (“Protocol A”) to the comparative protocol (“Protocol B”) that can be used in matching fresh frozen samples as described in Example 3.

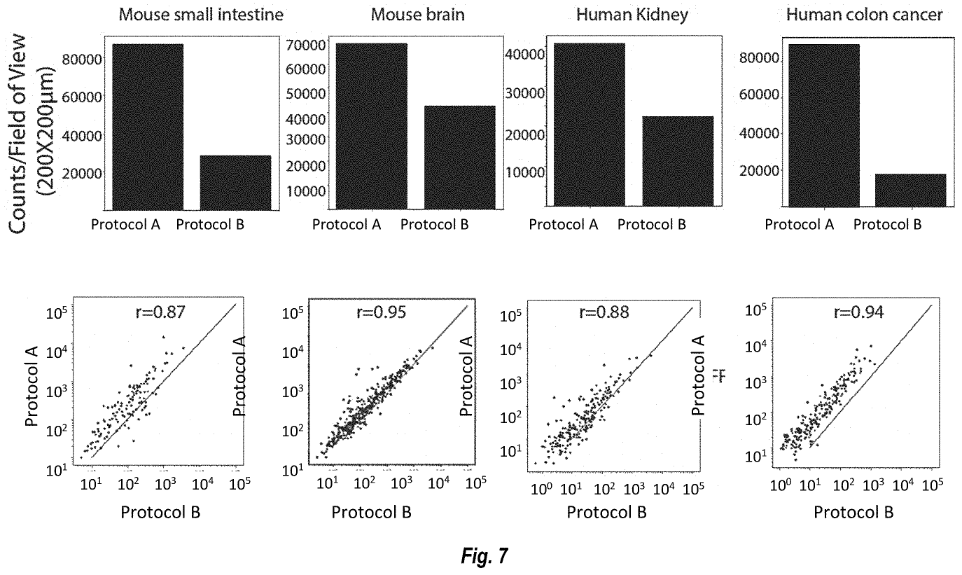

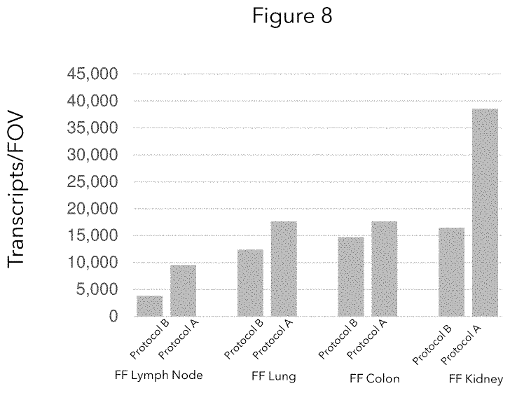

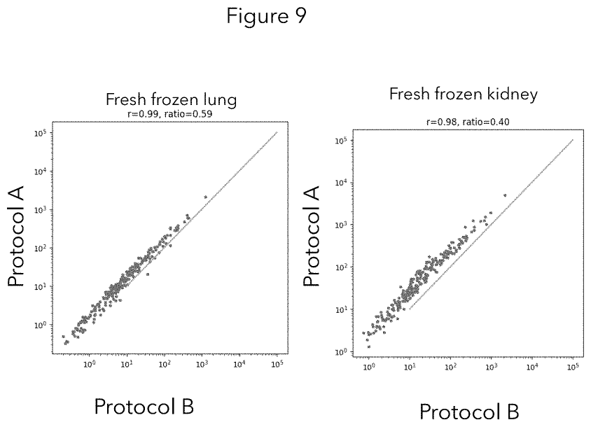

FIG. 7 shows that Protocol A provides high sensitivity and accuracy as compared to the comparative protocol (Protocol B) across different sample types. Average MERFISH counts per field of view with an area size of 200×200 μm were shown for the sample prepared by Protocol A and Protocol B across different tissue types (mouse small intestine, mouse brain, human kidney, and human colon cancer), with MERFISH counts with high correlation.

### Example 6. Single Cell Analysis in FFPE Human Melanoma Samples

This example demonstrates single-cell analysis in FFPE human melanoma samples. Cell segmentation was performed following antibody-based cell boundary staining as described in Example 1 above. Uniform Manifold Approximation and Projection (UMAP) clustering, a dimensionality reduction method that captures variability in a limited number of random variables to facilitate visualization of datasets with tens to thousands of dimensions, was used to define cell types in mixed populations based on the gene expression profile of individual cells and used for cell type identification.

The results showed spatial distributions of select cell types across the tissue and spatial distribution of select gene transcripts within the select cell types in melanoma were obtained. This shows that a spatial transcriptomics technique via MERFISH could be performed in the FFPE samples when prepared according to the methods described Example 1, with high detection efficiency and single molecule resolution, which eventually provided the gene expression profile in tissue samples in situ. Data not shown.

### Example 7. Single Cell Analysis in Various FFPE Samples

This example demonstrates single-cell analysis in various FFPE samples following the MERFISH protocol (Protocol A) according to the methods described in Example 1: A) Mouse small intestine. B) Mouse brain. C) Human liver cancer. D) Human kidney. E) Human lung. F) Human Ovarian cancer. G) Human uterus cancer. H) Human lung cancer. (Top) UMAP clustering for cell type identification; (Bottom) Spatial distribution of identified cell types.

The results indicate that when the FFPE sample was prepared according to the methods described in Example 1, it was possible to conduct in situ single-cell transcriptomic imaging of select gene transcripts in various tissue samples from human and mouse. Data not shown.

### Example 8. Immuno-Oncology Data Generated Using FFPE Samples

A summary of the data generated using FFPE on eight (8) sample types, 16 datasets, 500 genes, about 4 billion transcripts and about 9 million cells is shown in Table 1 below.

TABLE 1

| | Total | Total Cell | | |
|---|---|---|---|---|
| Sample Type | Transcripts | Number | RIN | DV200 |
| Colon cancer 1 | 411,716,053 | 677,451 | 2.4 | 72.56 |
| Colon cancer 2 | 507,576,479 | 817,588 | 3.3 | 87.54 |
| Liver cancer 1 | 272,021,991 | 568,355 | 2.1 | 76.97 |
| Liver cancer 2 | 283,068,068 | 598,141 | 3.3 | 71.31 |
| Melanoma 1 | 160,181,929 | 468,138 | 1.9 | 60.73 |
| Melanoma 2 | 75,617,432 | 207,869 | 1.8 | 65.64 |
| Ovarian cancer 1 | 197,365,319 | 358,485 | 4.4 | 75.75 |
| Ovarian cancer 2 | 121,798,559 | 254,347 | 4.5 | 74.82 |
| Ovarian cancer 3 | 32,054,444 | 71,381 | 4.5 | 74.82 |
| Ovarian cancer 4 | 119,742,527 | 212,425 | 4.5 | 74.82 |
| Prostate cancer 1 | 291,996,280 | 721,668 | 3.6 | 87.28 |
| Prostate cancer 2 | 221,331,615 | 993,825 | 3.2 | 79.56 |
| Lung cancer 1 | 144,388,044 | 353,762 | 4.3 | 66.97 |
| Lung cancer 2 | 425,594,806 | 836,739 | 2.8 | 80.97 |
| Breast cancer | 490,398,542 | 713,121 | 2.7 | 87.37 |
| Uterine cancer | 374,580,211 | 843,285 | 3.8 | 78.62 |
| Total | 4,129,432,299 | 8,696,580 | | |

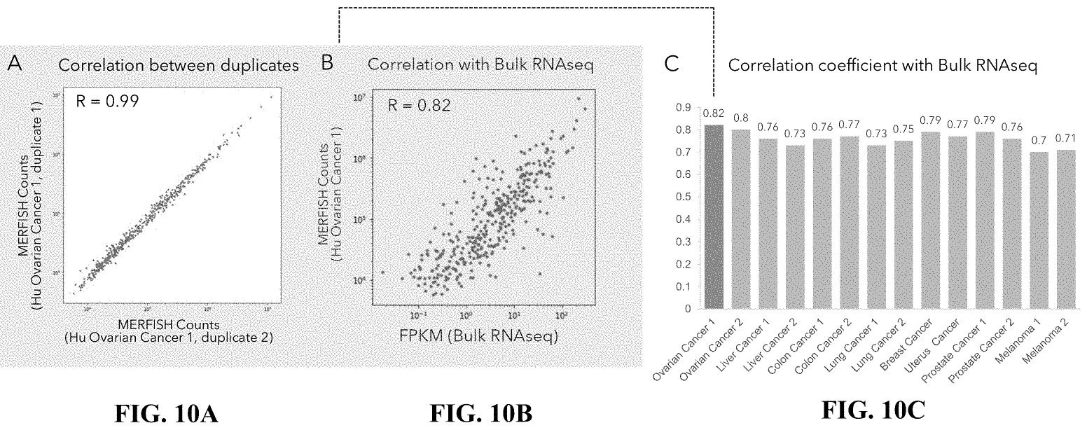

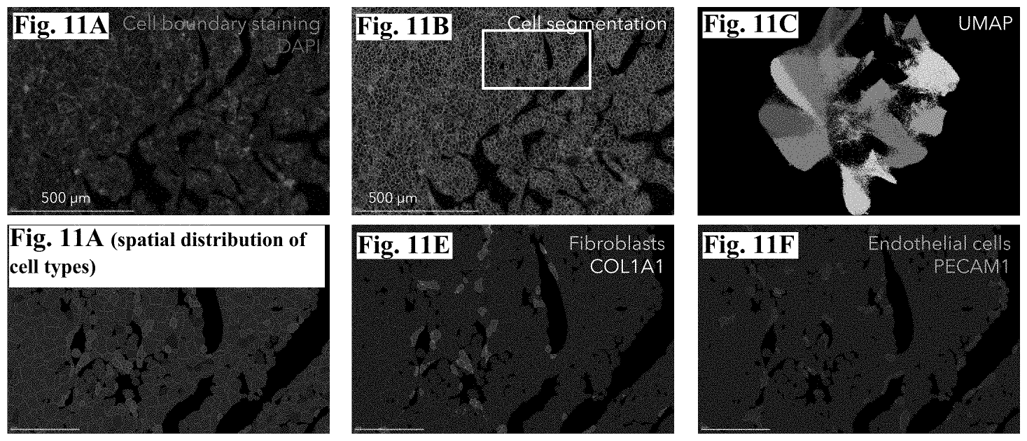

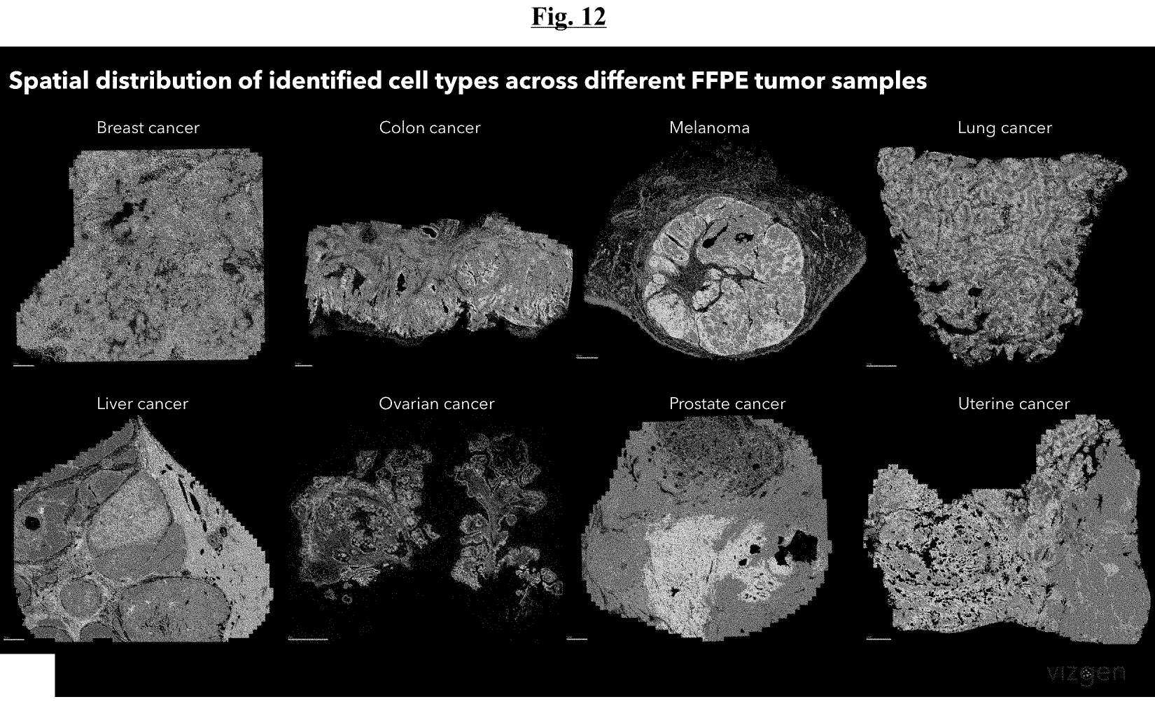

FIG. 10A-C show that the FFPE workflow is highly sensitive, accurate and reproducible. FIG. 10A shows the correlation of MERSCOPE data between two human ovarian cancer slices from the same patient. Correlation coefficient is 0.99, indicating the measurement is highly reproducible. In FIG. 10B, human ovarian cancer sample 1 was analyzed by MERSCOPE using a 500 gene panel, and adjacent slices were analyzed by bulk RNA sequencing. Correlation analysis between MERFISH counts and FPKM values from bulk RNA sequencing is shown. The correlation coefficient is 0.82, indicating the measurement is highly accurate. FIG. 10C presents a correlation analysis between MERSCOPE data and bulk RNA sequence was performed across 14 cancer samples, and correlation coefficients show high accuracy across multiple cancer types and replicates.

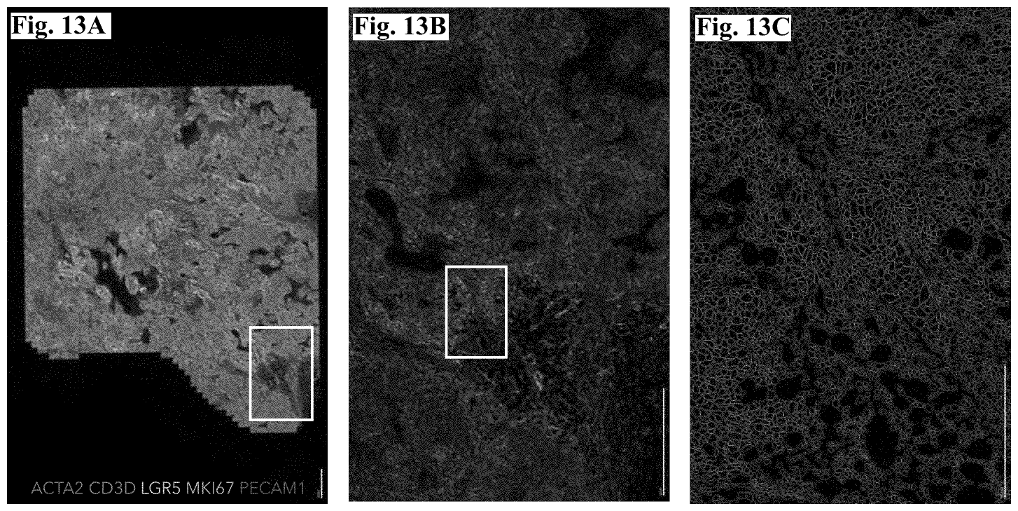

FIGS. 11A-F show that FFPE cell segmentation workflow enables true atlasing in dense tissue. In FIG. 11A, FFPE human liver cancer was immunostained with a cell boundary staining kit and DAPI for nucleus staining. In FIG. 11B, deep learning-based cell segmentation algorithm was used to segment cells. The polygon masks for each identified cell are shown. FIG. 11C shows UMAP visualization of 17 different cell types identified in human liver cancer generate from MERFISH transcript data. FIG. 11D shows the spatial distribution of identified cell types across the tissue in boxed region from FIG. 11B. FIG. 11E shows spatial distribution of fibroblasts in boxed region from FIG. 11B. Fibroblast marker gene COL1A1. FIG. 11F shows the partial distribution of endothelial cells in boxed region from B. Endothelial marker gene PECAM1.

FIG. 12 shows the spatial distribution of identified cell types across different FFPE tumor samples. Different cancer samples, including breast cancer, colon cancer, melanoma, lung cancer, liver cancer, ovarian cancer, prostate cancer and uterine cancer, were analyzed by MERSCOPE using a 500 gene panel, together with cell boundary staining kit to label the cell boundary. Cells were segmented and subjected for single cell analysis. Identified cells in each sample were colored to show the spatial distribution of different cells across the sample. Scale bar: 1 mm.

FIG. 13 shows that the FFPE protocol can be used to show the spatial distribution of the expression of select genes (ACTA2, CD3D, LGR5, MK167, and PECAM1) in human breast cancer. FIG. 13A shows the spatial distribution of select genes including ACTA2 (green), CD3D (red), LGR5 (light green), MKI67 (magenta) and PECAM1 (blue) from 500 genes analyzed across the tissue. Scale bar: 1 mm. FIG. 13B provides a zoomed-in view of the boxed region in FIG. 13A. Scale bar: 1 mm. FIG. 13C shows a zoom-in view of the boxed region in FIG. 13B, with cell boundary polygon masks shown in grey. Scale bar: 250 μm.

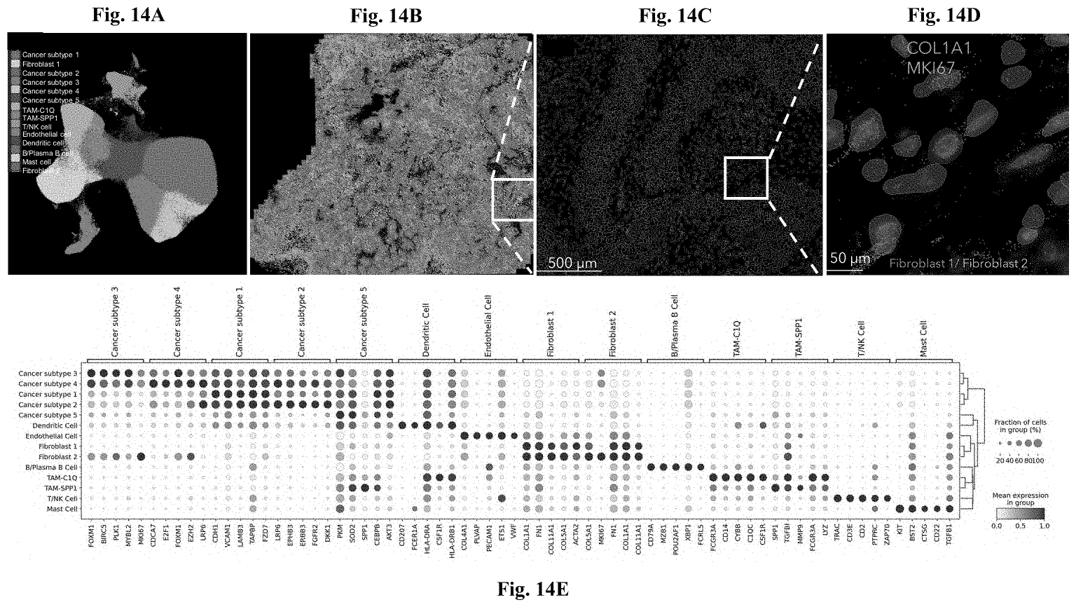

FIG. 14A-E show that the FFPE protocol can be used for cell type identification and mapping in human breast cancer. FIG. 14A provides UMAP visualization of different cell types identified in human breast cancer generated from MERFISH transcript data. FIG. 14B shows the spatial distribution of 14 identified cell types across the tissue. FIG. 14C shows the spatial distribution of identified cell types in boxed region in FIG. 14B. FIG. 14D shows the spatial distribution of two types of fibroblasts (fibroblast 1 in green and fibroblast 2 in red) in boxed region in FIG. 14C. Both types of fibroblasts express COL1A1 gene, while fibroblast 2 expresses proliferation marker MKI67. FIG. 14E provides a dot plot showing the marker genes for each cell type.

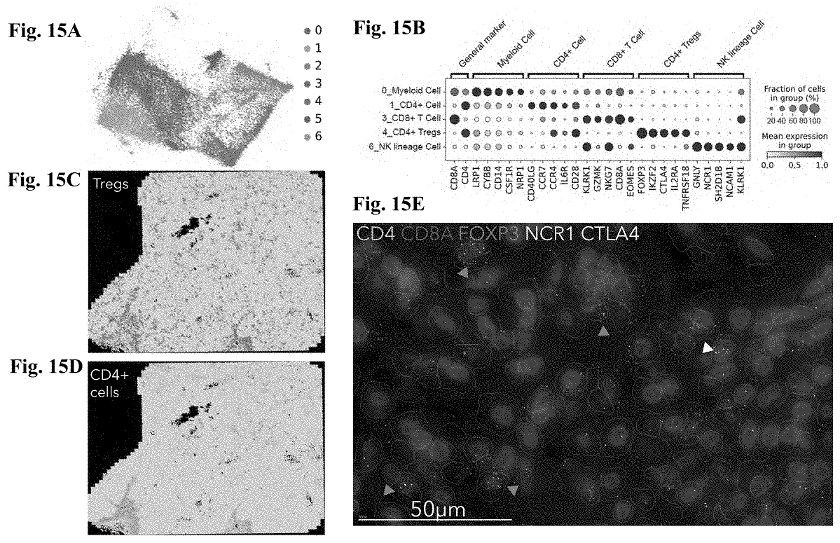

FIG. 15 shows that the FFPE protocol can be used to characterize immune cell types in the tumor microenvironment. FIG. 15A shows the T/NK cell cluster from a breast cancer sample was selected for sub-clustering analysis. UMAP visualization of sub-clustering analysis showing 7 different immune cell subtypes within human breast cancer. FIG. 15B provides a dot plot showing the marker genes for each immune cell type, including Myeloid cells, CD4+ T cells, CD8+ T cells, CD4+ regulatory T cells (Tregs), and NK lineage cells. FIG. 15C provides the spatial distribution of Tregs. FIG. 15D provides the spatial distribution of CD4+ T cells. FIG. 15E provides the spatial distribution of select genes within a magnified region in human breast cancer, with CD4 (green), CD8A (blue), FOXP3 (red), NCR1 (yellow) and CTLA4 (white) shown. Note that FOXP3 positive Tregs expresses T cell exhaustion marker (CTLA4 marked by red arrowhead; NK cell marked by yellow arrowhead).

1. A method of anchoring target nucleic acid within a matrix and clearing non-target cellular components comprising:
a. contacting a formalin fixed paraffin embedded (FFPE) tissue sample with at least two anchoring agents, wherein the first anchoring agent forms a covalent bond with the target nucleic acid and the second anchoring agent comprises an oligonucleotide that hybridizes with the target nucleic acid;
b. embedding the sample in a polymer matrix wherein the first and second anchoring agents each form a covalent bond with the polymer matrix; and,
c. clearing the non-target cellular components from the polymer matrix wherein the target nucleic acid remains anchored in the polymer matrix to form a matrix anchored target nucleic acid sample.

2. The method of claim 1, wherein the first anchoring agent is an alkylating agent.

3. The method of claim 2, wherein the alkylating agent is selected from the group consisting of Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa and Trabectedin.

4. The method of claim 1, wherein the second anchoring agent comprises alternating dT and locked dT portions that hybridizes with the target nucleic acid.

5. The method of claim 1, wherein the second anchoring agent comprises a poly-dT portion that hybridizes to the target nucleic acid.

6. The method of claim 1, wherein the first anchoring agent and the second anchoring agent each comprise an acrydite moiety that covalently binds the polymer matrix.

7. The method of claim 1, wherein the first anchoring agent is an alkylating agent derivatized with an acrydite moiety.

8. The method of claim 1, wherein the second anchoring agent comprises a poly-dT portion that hybridizes to the target nucleic acid and an acrydite moiety that covalently binds the polymer matrix.

9. The method of claim 1, wherein the target nucleic acid is RNA.

10. The method of claim 1, wherein the target nucleic acid is DNA.

11. The method of claim 1, further comprising contacting the anchored target nucleic acid sample with one or more primary oligonucleotide probes that hybridize to the target nucleic acids.

12. The method of claim 11, wherein the primary oligonucleotide probes are single molecule (sm)FISH probes or multiplexed error robust fluorescence in situ hybridization (MERFISH) probes.

13. The method of claim 11, wherein the one or more primary oligonucleotide probes comprise a first portion comprising a target sequence and a second portion comprising one or more read sequences.

14. The method of claim 13, further comprising determining read sequences based on contacting the one or more primary oligonucleotide probes with a plurality of secondary nucleic acid probes comprising a recognition sequence that hybridizes to the read sequence of the primary nucleic acid probe.

15. The method of claim 1, further comprising imaging using multiplexed fluorescence in situ hybridization comprising contacting the anchored target nucleic acid sample with one or more primary oligonucleotide probes that hybridize to the target nucleic acids and comprising one or more sequential steps of adding a plurality of secondary nucleic acid probes comprising a label moiety.

16. The method of claim 1, further comprising imaging using multiplexed error robust fluorescence in situ hybridization (MERFISH) probes comprising contacting the anchored target nucleic acid sample with one or more MERFISH probes that hybridize to the target nucleic acids.

17. The method of claim 13, further comprising imaging using multiple rounds of fluorescence in situ hybridization wherein, in each round, one or more different secondary nucleic acid probes, each conjugated to a spectrally distinct fluorescent label are used to readout out multiple readout sequences simultaneously.

18. A method of anchoring target RNA within a matrix and clearing non-target cellular components comprising:
a. contacting a formalin fixed paraffin embedded (FFPE) tissue sample with at least two anchoring agents, wherein the first anchoring agent comprises an alkylating agent that forms a covalent bond with the target nucleic acid and the second anchoring agent comprises a polyT sequence that is complementary and hybridizes to the target RNA and wherein the first anchoring agent and the second anchoring agent each comprise an acrydite moiety that covalently binds to the matrix;
b. embedding the sample in a polymer matrix wherein the first and second anchoring agents each form a covalent bond with the polymer matrix; and,
c. clearing the non-target cellular components from the polymer matrix wherein the target RNA remains anchored in the polymer matrix to form a matrix anchored target RNA sample.

19. The method of claim 18, wherein the alkylating agent is selected from the group consisting of Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa and Trabectedin.

20-27. (canceled)

28. A method for imaging target nucleic acid within a matrix and clearing non-target cellular components comprising:
a. contacting a formalin fixed paraffin embedded (FFPE) tissue sample with at least two anchoring agents, wherein the first anchoring agent forms a covalent bond with the target nucleic acid and the second anchoring agent comprises an oligonucleotide that hybridizes with the target nucleic acid;
b. embedding the sample in a polymer matrix wherein the first and second anchoring agents each form a covalent bond with the polymer matrix;
c. clearing the non-target cellular components from the polymer matrix wherein the target nucleic acid remains anchored in the polymer matrix to form a matrix anchored target nucleic acid sample; and,
d. contacting the anchored target nucleic acid sample with one or more primary oligonucleotide probes that hybridize to the target nucleic acids and a plurality of secondary nucleic acid probes comprising a fluorescent label and a recognition sequence that hybridizes to a sequence of the primary nucleic acid probe and imaging the target nucleic acids.

29-77. (canceled)
